(19) United States
(12) Patent Application Publication
(10) Pub. No.: US 2023/0043576 A1
(43) Pub. Date: Feb. 9, 2023

(54) METHODS OF TREATING PREGNANCY-ASSOCIATED ATYPICAL HEMOLYTIC UREMIC SYNDROME USING AN ANTI-C5 ANTIBODY

(71) Applicant: Alexion Pharmaceuticals, Inc., Boston, MA (US)

(72) Inventor: Andrew E. Denker, Ardmore, PA (US)

(73) Assignee: Alexion Pharmaceuticals, Inc., Boston, MA (US)

(21) Appl. No.: 17/784,025

(22) PCT Filed: Dec. 18, 2020
(86) PCT No.: PCT/US2020/065924

§ 371 Date: Jun. 9, 2022

Related U.S. Application Data
(60) Provisional application No. 62/952,971, filed on Dec. 23, 2019.
(60) Provisional application No. 63/007,489, filed on Apr. 9, 2020.
(60) Provisional application No. 62/704,879, filed on Jun. 1, 2020.

(51) Int. Cl. C07K 16/18; A61K 9/00; A61P 7/00

(52) U.S. Cl. CPC C07K 16/18; A61K 9/0019; A61P 7/00; C07K 2317/565; C07K 2317/92; C07K 2317/526

(57) ABSTRACT

Provided are methods for clinical treatment of pregnancy-associated atypical haemolytic uraemic syndrome (p-aHUS), including postpartum aHUS, using an anti-C5 antibody, or antigen binding fragment thereof, such as ravulizumab (ULTOMIRIS®).

### CROSS REFERENCE TO RELATED APPLICATIONS

This application claims the benefit of U.S. Provisional Application No. 62/952,971, filed Dec. 23, 2019, U.S. Provisional Application No. 63/007,489, filed Apr. 9, 2020, and U.S. Provisional Application No. 62/704,879, filed Jun. 1, 2020, each of which is incorporated by reference herein in its entirety.

### BACKGROUND

Hemolytic uremic syndrome (HUS) is characterized by thrombocytopenia, microangiopathic hemolytic anemia, and acute renal failure. HUS is classified as one of two types: diarrheal-associated (D+ HUS; also referred to as shiga toxin producing E. coli (STEC)-HUS or typical HUS) and non-diarrheal or atypical HUS (aHUS). D+ HUS is the most common form, accounting for greater than 90% of cases and is caused by a preceding illness with a shiga-like toxin-producing bacterium, e.g., E. coli O157:H7.

aHUS can be genetic, acquired, or idiopathic. Hereditable forms of aHUS can be associated with mutations in a number of human complement components including, e.g., complement factor H (CFH), membrane cofactor protein (MCP), complement factor I (CFI), C4b-binding protein (C4BP), complement factor B (CFB), and complement component 3 (C3) (see, e.g., Caprioli et al. (2006) Blood 108:1267-1279). Certain mutations in the gene encoding CD55, though not yet implicated in aHUS, are associated with the severity of aHUS (see, e.g., Esparza-Gordillo et al. (2005) Hum Mol Genet 14:703-712).

aHUS triggered by pregnancy (“p-aHUS”) is a rare and severe systemic disease associated with dysregulation of the alternative complement pathway that occurs in approximately 1 in 25,000 pregnancies. Hyperactivation of complement results in diffuse endothelial injury with subsequent formation of fibrin and platelet microthrombi in the vasculature, which leads to hemolysis, thrombocytopenia, and end organ dysfunction from ischemia (primarily in the form of acute kidney injury) (see, e.g., Saad, et al., AJP Reports vol. 6,1 (2016)). The majority of cases of p-aHUS occur during the postpartum period and are known as postpartum aHUS (see, e.g., Fakhouri F, et al., J. Am. Soc. Nephrol. 2010; 21(5):859-867).

Patients with p-aHUS (including postpartum aHUS) are at risk of substantial morbidity and mortality. Accordingly, it is an object of the present invention to provide improved methods for treating patients with p-aHUS.

### SUMMARY

Provided herein are compositions and methods for treating pregnancy-associated atypical haemolytic uraemic syndrome (p-aHUS) (e.g., postpartum aHUS) in a human patient, comprising administering to the patient an anti-C5 antibody, or antigen binding fragment thereof (e.g., ravulizumab). In some embodiments, the anti-C5 antibody, or antigen binding fragment thereof, is administered (or is for administration) according to a particular clinical dosage regimen (i.e., at a particular dose amount and according to a specific dosing schedule). In some embodiments, the patient has severe or early onset p-aHUS. In some embodiments, the patient also has preeclampsia (PE) and/or HELLP syndrome.

Any suitable anti-C5 antibody, or antigen binding fragment thereof, can be used in the methods described herein. An exemplary anti-C5 antibody is eculizumab. Eculizumab (also known as SOLIRIS®) is an anti-C5 antibody comprising heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. Eculizumab comprises a heavy chain variable region having the amino acid sequence set forth in SEQ ID NO: 7 and a light chain variable region having the amino acid sequence set forth in SEQ ID NO: 8. Eculizumab comprises a heavy chain comprising the amino acid sequence set forth in SEQ ID NO:10 and a light chain having the amino acid sequence set forth in SEQ ID NO:11.

Another exemplary anti-C5 antibody is ravulizumab (also known as ULTOMIRIS®, ALXN1210 and antibody BNJ441) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab.

Accordingly, in one embodiment, the antibody comprises the CDR1, CDR2, and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fe constant region, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

Another exemplary anti-C5 antibody is described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region having the sequence set forth in SEQ ID NO:27, and the VL region having the sequence set forth in SEQ ID NO:28.

Another exemplary anti-C5 antibody is also described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region having the sequence set forth in SEQ ID NO: 35, and the VL region having the sequence set forth in SEQ ID NO: 36.

Another exemplary anti-C5 antibody is described in US2016/0176954A1. In one embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region having the sequence set forth in SEQ ID NO: 43, and the VL region having the sequence set forth in SEQ ID NO: 44.

Another exemplary anti-C5 antibody is described in Fukuzawa T. et al. (Sci. Rep. 7:1080, 2017). In another embodiment, the antibody, or antigen binding fragment thereof, comprises a heavy chain comprising SEQ ID NO: 45 and a light chain comprising SEQ ID NO: 46.

Another exemplary anti-C5 antibody is described in US20170355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 91%, 92%, 93%, 94%, 95%, 96%, 97%, 98% or 99% variable region identity).

In one embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5 and 6, respectively. In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises a heavy chain variable region having the amino acid sequence set forth in SEQ ID NO: 7 and a light chain variable region having the amino acid sequence set forth in SEQ ID NO: 8. In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises a heavy chain comprising the amino acid sequence set forth in SEQ ID NO:10 and a light chain having the amino acid sequence set forth in SEQ ID NO:11.

In one embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody further comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In one embodiment, the anti-C5 antibody, or antigen binding fragment is administered at a fixed dose. For example, in one embodiment, the anti-C5 antibody, or antigen binding fragment is administered at a dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg, without regard to the patient's weight. In another embodiment, the anti-C5 antibody, or antigen binding fragment is administered at a sub-therapeutic dose.

In another embodiment, the anti-C5 antibody, or antigen binding fragment thereof, (e.g., ravulizumab) is administered at a dose of 2400 mg, 2700 mg, 3000 mg, 3300 mg, or 3600 mg.

In another embodiment, the dose of the anti-C5 antibody, or antigen binding fragment is based on the weight of the patient. For example, in one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg every two weeks. In another embodiment, 2400 mg or 3000 mg of the anti-C5 antibody, or antigen binding fragment thereof, (e.g., ravulizumab) is administered to a patient weighing ≥40 to <60 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment thereof, is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg every two weeks. In another embodiment, 2700 mg or 3300 mg of the anti-C5 antibody, or antigen binding fragment thereof, (e.g., ravulizumab) is administered to a patient weighing ≥60 to <100 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment, is administered to a patient weighing ≥100 kg. In another embodiment, 3000 mg or 3600 mg of the anti-C5 antibody, or antigen binding fragment thereof, (e.g., ravulizumab) is administered to a patient weighing ≥100 kg.

In another embodiment, a method of treating a human patient with p-aHUS is provided, the method comprising administering to the patient an anti-C5 antibody, or antigen binding fragment thereof (e.g., ravulizumab):
(a) once on Day 1 at a dose of: 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In another embodiment, a method of treating a human patient with p-aHUS is provided, wherein the anti-C5 antibody, or antigen binding fragment thereof, (e.g., ravulizumab) is administered to a patient weighing ≥40 to <60 kg:
(a) once on Day 1 at a dose of 2400 mg; and
(b) on Day 15 and every eight weeks thereafter at a dose of 3000 mg.

In another embodiment, a method of treating a human patient with p-aHUS is provided, wherein the anti-C5 antibody, or antigen binding fragment thereof, (e.g., ravulizumab) is administered to a patient weighing ≥60 to <100 kg:
(a) once on Day 1 at a dose of 2700 mg; and
(b) on Day 15 of the administration cycle and every eight weeks thereafter at a dose of 3300 mg.

In another embodiment, a method of treating a human patient with p-aHUS is provided, wherein the anti-C5 antibody, or antigen binding fragment thereof, (e.g., ravulizumab) is administered to a patient weighing ≥100 kg:
(a) once on Day 1 at a dose of 3000 mg; and
(b) on Day 15 and every eight weeks thereafter at a dose of 3600 mg.

In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fc receptor (FcRn), wherein the variant human Fe CH3 region comprises Met-429-Leu and Asn-435-Ser substitutions at residues corresponding to methionine 428 and asparagine 434 of a native human IgG Fe region, each in EU numbering, and wherein the anti-C5 antibody, or antigen binding fragment thereof, is administered to the patient: (a) once on Day 1 at a dose of: 2400 mg to a patient weighing ≥40 to <60 kg, 2700 mg to a patient weighing ≥60 to <100 kg, or 3000 mg to a patient weighing ≥100 kg; and (b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In another embodiment, the anti-C5 antibody, or antigen binding fragment, is administered at a milligram per kilogram (mg/kg) dose. For example, in one embodiment, the anti-C5 antibody, or antigen binding fragment thereof, is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg, or 25.0 mg/kg.

In one embodiment, the anti-C5 antibody, or antigen binding fragment is administered once per week, twice per week, three times per week, four times per week, five times per week, six times per week, or daily. In another embodiment, the anti-C5 antibody, or antigen binding fragment, is administered twice daily. In another embodiment, anti-C5 antibody, or antigen binding fragment, is administered once every two weeks, once every three weeks, once every four weeks, once every five weeks, once every six weeks, once every seven weeks, once every eight weeks, once every nine weeks, once every ten weeks, once every eleven weeks, or once every twelve weeks. In another embodiment, the anti-C5 antibody, or antigen binding fragment, is administered at a loading dose on Day 1, followed by a different maintenance dose on Day 15 and every eight weeks thereafter.

In other embodiments, the anti-C5 antibody, or antigen binding fragment thereof, is administered immediately after symptoms first occur. In other embodiments, treatment begins 1-20 days after symptoms first occur. In other embodiments, treatment begins 1, 2, 3, 4, 5, 6, 7, 8, 9, 10, 11, 12, 13, 14, 15, 16, 17, 18, 19, or 20 days after symptoms first occur. In other embodiments, treatment begins 5-15 days after delivery. In other embodiments, treatment begins 1, 2, 3, 4, 5, 6, 7, 8, 9, 10, 11, 12, 13, 14, 15, 16, 17, 18, 19, or 20 days after delivery.

In another embodiment, the anti-C5 antibody, or antigen binding fragment thereof, is administered for one or more administration cycles. In one embodiment, the administration cycle is 26 weeks. In another embodiment, the treatment comprises at least 1, 2, 3, 4, 5, 6, 7, 8, 9, 10, or 11 cycles. In another embodiment, the patient is treated for about 1, 2, 3, 4, 5, or 6 months. In another embodiment, the treatment continues for the lifetime of the human patient.

In some embodiments, the patient has not previously been treated with a complement inhibitor (e.g., the patient is a complement inhibitor treatment-naïve patient). In another embodiment, the patient is a complement inhibitor treatment-naïve patient who has previously received plasma exchange and/or dialysis.

The anti-C5 antibody, or antigen binding fragment, can be administered via any suitable means. In one embodiment, the anti-C5 antibody, or antigen binding fragment (e.g., ravulizumab), is administered intravenously. In another embodiment, the anti-C5 antibody, or antigen binding fragment, is administered subcutaneously.

In another aspect, methods of treating a human patient with p-aHUS are provided, the method comprising administering to the patient an effective amount of a first anti-C5 antibody, or antigen binding fragment thereof (e.g., ravulizumab), followed by a second anti-C5 antibody or antigen-binding fragment.

In some embodiments, the patients treated according to the methods described herein have been vaccinated against meningococcal infections within 3 years prior to, or at the time of, initiating treatment. In one embodiment, patients who received treatment less than 2 weeks after receiving a meningococcal vaccine are also treated with appropriate prophylactic antibiotics until 2 weeks after vaccination. In another embodiment, patients treated according to the methods described herein are vaccinated against meningococcal serotypes A, C, Y, W135, and/or B.

In another aspect, the treatment regimens described are sufficient to maintain particular serum trough concentrations of the anti-C5 antibody, or antigen binding fragment thereof. For example, in one embodiment, the treatment can maintain a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/mL or greater. In one embodiment, the treatment maintains a serum trough concentration of the anti-C5 antibody, or antigen binding fragment thereof, of 100 μg/mL or greater. In another embodiment, the treatment maintains a serum trough concentration of the anti-C5 antibody, or antigen binding fragment thereof, of 150 μg/mL or greater. In another embodiment, the treatment maintains a serum trough concentration of the anti-C5 antibody, or antigen binding fragment thereof, of 200 μg/mL or greater. In another embodiment, the treatment maintains a serum trough concentration of the anti-C5 antibody, or antigen binding fragment thereof, of 250 μg/mL or greater. In another embodiment, the treatment maintains a serum trough concentration of the anti-C5 antibody, or antigen binding fragment thereof, of 300 μg/mL or greater. In another embodiment, the treatment maintains a serum trough concentration of the anti-C5 antibody, or antigen binding fragment thereof, of between 100 μg/ml and 200 μg/mL. In another embodiment, the treatment maintains a serum trough concentration of the anti-C5 antibody, or antigen binding fragment thereof, of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. For example, in one embodiment, the anti-C5 antibody can be administered to the patient in an amount and with a frequency to maintain a free C5 concentration of 0.2 μg/mL, 0.3 μg/mL, 0.4 μg/mL, 0.5 μg/mL or below. In another embodiment, the treatment described herein reduces free C5 concentration by greater than 99% throughout the treatment period.

In another aspect, the methods of treating p-aHUS described herein can be used alone or in combination with one more additional therapies and/or therapeutic agents. For example, in one embodiment, the method further comprises administering to the patient an anti-inflammatory agent (e.g., prednisone).

The efficacy of the treatment methods provided herein can be assessed using any suitable means. In one embodiment, the treatment results in produces at least one therapeutic effect selected from the group consisting of a reduction or cessation in severe hypertension, proteinuria, uremia, lethargy/fatigue, irritability, thrombocytopenia, microangiopathic hemolytic anemia, and renal function impairment (e.g., acute renal failure) compared to baseline.

In other embodiments, the treatment results in terminal complement inhibition.

In other embodiments, the treatment produces a shift toward normal levels of a hemolysis-related hematologic biomarker selected from the group consisting of free hemoglobin, haptoglobin, reticulocyte count, PNH red blood cell (RBC) clone and D-dimer compared to baseline.

In another embodiment, the treatment produces a shift toward normal levels of Factor Ba, soluble tumor necrosis factor receptor 1 [sTNFR1]), soluble vascular adhesion molecule 1 [sVCAM1], thrombomodulin, D-dimer, and cystatin C.

In another embodiment, the treatment produces an increase in hemoglobin stabilization compared to the patient's pre-treatment baseline. In another embodiment, the treatment results in a ≥20 g/L increase in hemoglobin.

In other embodiments, the treatment results in platelet normalization (≥150×10⁹/L). In other embodiments, the treatment results in platelet normalization (≥150×10⁹/L) for at least 28 days (e.g., at least 28 days, 1 month, 2 months, 3 months, 4 months, 5 months, 6 months, 7 months, 8 months, 9 months, 10 months, 11 months, 1 year, or two years). In other embodiments, the treatment results in platelet normalization 5-12 days after initiating treatment. For example, in one embodiment, platelet normalization occurs 5, 6, 7, 8, 9, 10, 11, or 12 days after initiating treatment. In a particular embodiment, platelet normalization occurs 8 days after initiating treatment.

In other embodiments, the treatment results in LDH normalization (≤246 U/L). In other embodiments, the treatment results in LDH normalization (≤246 U/L) for at least 28 days (e.g., at least 28 days, 1 month, 2 months, 3 months, 4 months, 5 months, 6 months, 7 months, 8 months, 9 months, 10 months, 11 months, 1 year, or two years). In other embodiments, the treatment results in LDH normalization 5-12 days after initiating treatment. For example, in one embodiment, LDH normalization occurs 5, 6, 7, 8, 9, 10, 11, or 12 days after initiating treatment. In another embodiment, LDH normalization occurs 8 days after initiating treatment. In another embodiment, LDH and platelet normalization occurs 8 days after initiating treatment.

In other embodiments, the treatment results in a 25% improvement from baseline in serum creatinine. In other embodiments, the treatment results in a 25% improvement from baseline in serum creatinine for at least 28 days (e.g., at least 28 days, 1 month, 2 months, 3 months, 4 months, 5 months, 6 months, 7 months, 8 months, 9 months, 10 months, 11 months, 1 year, or two years).

In other embodiments, the treatment results in a complete TMA response (i.e., platelet normalization (≥150×10⁹/L), LDH normalization (246 U/L), and a 25% improvement from baseline in serum creatinine). In other embodiments, the treatment results in a complete TMA response for at least 28 days (e.g., at least 28 days, 1 month, 2 months, 3 months, 4 months, 5 months, 6 months, 7 months, 8 months, 9 months, 10 months, 11 months, 1 year, or two years). In other embodiments, the treatment confers complete TMA response in the patient in fewer than about 57 days (e.g., 56, 55, 54, 53, 52, 51, 50, 49, 48, 48, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day(s)). In other embodiments, the treatment confers complete TMA response in the patient in fewer than about 43 days. In other embodiments, the treatment confers complete TMA response in the patient in fewer than about 22 days. In other embodiments, the treatment confers complete TMA response in the patient in fewer than about 15 days. In other embodiments, treatment of p-aHUS patients with the anti-C5 antibody, or antigen binding fragment thereof, (e.g., ravulizumab) confers complete TMA response in about 32 days (range: 8 days to 57 days, with 95% CI between 9 days and 46 days).

In other embodiments, the treatment results in a modified complete TMA Response (i.e., platelet normalization (≥150×10⁹/L), LDH normalization (246 U/L), and the patient is off dialysis if they were on dialysis at baseline or a 25% improvement from baseline in serum creatinine for a patient who was off dialysis at baseline). In other embodiments, the treatment results in a modified complete TMA Response for at least 28 days (e.g., at least 28 days, 1 month, 2 months, 3 months, 4 months, 5 months, 6 months, 7 months, 8 months, 9 months, 10 months, 11 months, 1 year, or two years).

In other embodiments, the treatment results in hematological normalization comprising platelet count normalization (e.g., ≥150×109/L), lactate dehydrogenase (LDH) normalization (e.g., <246 U/L), preferably both platelet count and LDH normalization, optionally together with improvement in serum creatinine (e.g., 25% improvement) over time (e.g., 2 separate assessments 28 days apart).

In other embodiments, the treatment produces a reduction in the need for blood transfusions. In another embodiment, the treatment produces a greater than 70% increase in transfusion avoidance.

In other embodiments, the treatment results in an elimination of breakthrough hemolysis during the treatment period. In another embodiment, the treatment results in a reduction of breakthrough hemolysis compared to pretreatment baseline amount of breakthrough hemolysis.

In other embodiments, the treatment produces a reduction in major adverse vascular events (MAVEs).

In other embodiments, the treatment improves dialysis requirement status compared to baseline, e.g., a reduction or total discontinuation of dialysis. In one embodiment, the treatment results in a discontinuation of dialysis within 15 to 30 days. For example, in other embodiments, dialysis is discontinued within 15, 16, 17, 18, 19, 20, 21, 22, 23, 24, 25, 26, 27, 28, 29, or 30 days after initiating treatment. In one embodiment, dialysis is discontinued within about 21 days after initiating treatment.

In other embodiments, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale. In one embodiment, the treatment produces a change from baseline in quality of life as assessed via the FACIT-Fatigue Scale by one or more (e.g., 1, 2, or 3) points. In another embodiment, the treatment produces a change from baseline in quality of life as assessed via the FACIT-Fatigue Scale by 3 points, 150 days or more (e.g., 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 205 days, 210 days, 215 days, 220 days, or 225 days) after initiating treatment.

In other embodiments, the treatment results in a shift towards normal levels of eGFR (e.g., ≥90). In other embodiments, the treatment improves eGFR status compared to baseline. In other embodiments, the treatment improves eGFR status compared to baseline within 5-10 days of initiating treatment. For example, in one embodiment, the treatment improves eGFR status compared to baseline within 5, 6, 7, 8, 9, or 10 days of initiating treatment. In another embodiment, the treatment improves eGFR status compared to baseline within 8 days of initiating treatment.

In other embodiments, the treatment prolongs pregnancy (e.g., by days, weeks, or months). For example, in some embodiments, the treatment prolongs pregnancy by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In other embodiments, the treatment prolongs pregnancy by 1, 2, 3, 4, 5, 6, 7, 8, 9, 10, 11, 12, 13, 14, 15, 16, 17, 18, 19, or 20 weeks. In other embodiments, the treatment prolongs pregnancy by 1, 2, 3, 4, or 5 months. In other embodiments, the treatment advances gestational age (e.g., by days, weeks, or months). For example, in some embodiments, the treatment advances gestational age by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In other embodiments, the treatment advances gestational age by 1, 2, 3, 4, 5, 6, 7, 8, 9, 10, 11, 12, 13, 14, 15, 16, 17, 18, 19, or 20 weeks. In other embodiments, the treatment advances gestational age by 1, 2, 3, 4, or 5 months.

In other embodiments, the treatment prevents end-stage renal disease (ESRD). In other embodiments, the treatment prolongs time to ESRD (e.g., by days, weeks, months, or years), for example, at least by 10 months, 20 months, 40 months, 60 months, 80 months, 100 months, or at least by 120 months. In some embodiments, the treatment prolongs time to ESRD after initial manifestation of thrombotic microangiopathies (TMA).

In other embodiments, the treatment prolongs survival of the patient (e.g., by days, weeks, months, or years), for example, at least by 10 months, 20 months, 40 months, 60 months, 80 months, 100 months, or at least by 120 months.

Further provided are kits for treating p-aHUS (e.g., postpartum aHUS). In one embodiment, the kit comprises (a) a dose of an anti-C5 antibody, or antigen binding fragment thereof (e.g., any of those previously described herein); and (b) instructions for using the anti-C5 antibody, or antigen binding fragment thereof, in the methods described herein.

In one embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5 and 6, respectively; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises a heavy chain variable region having the amino acid sequence set forth in SEQ ID NO: 7 and a light chain variable region having the amino acid sequence set forth in SEQ ID NO: 8; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises a heavy chain comprising the amino acid sequence set forth in SEQ ID NO:10 and a light chain having the amino acid sequence set forth in SEQ ID NO:11; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of eculizumab and (b) instructions for using eculizumab in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fec receptor (FcRn), wherein the variant human Fc CH3 region comprises Met-429-Leu and Asn-435-Ser substitutions at residues corresponding to methionine 428 and asparagine 434 of a native human IgG Fec region, each in EU numbering; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of ravulizumab and (b) instructions for using ravulizumab in the methods described herein.

### BRIEF DESCRIPTION OF THE DRAWING

FIG. 1 is a Kaplan-Meier graph that depicts the median time to complete TMA response in days for eight patients with postpartum-aHUS enrolled in a phase 3, multi-center study (“311 Study”).

FIG. 2 is a graph depicting the observed platelet count value over time. Data are shown as mean (error bars, 95% confidence interval). “BL” is baseline.

FIG. 3 is a graph depicting observed lactate dehydrogenase values over time. Data are shown as mean (error bars, 95% confidence interval). “BL” is baseline.

FIG. 4 is a graph depicting observed eGFR values over time. Data are shown as mean (error bars, 95% confidence interval). “BL” is baseline. “eGFR” is estimated glomerular filtration rate.

FIG. 5 is a Kaplan-Meier estimation graph of survival using time to end-stage renal disease (ESRD) after initial TMA manifestation.

### DETAILED DESCRIPTION

### I. Anti-C5 Antibodies

The anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

The term “antibody” describes polypeptides comprising at least one antibody derived antigen binding site (e.g., V_{H}/V_{L} region or F_{y}, or CDR). Antibodies include known forms of antibodies. An antibody can be, for example, a human antibody, a humanized antibody, a bispecific antibody or a chimeric antibody. An antibody also can be a Fab, Fab′2, ScFv, SMIP, Affibody®, nanobody or a domain antibody. An antibody also can be of any of the following isotypes: IgG1, IgG2, IgG3, IgG4, IgM, IgA1, IgA2, IgAsec, IgD, IgE or a hybrid of any of these isotypes. An antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody), which changes a property (e.g., a functional property) of the antibody. Numerous such alterations are known in the art that affect, e.g., half-life, effector function, and/or immune responses to the antibody in a patient. The term “antibody” also includes artificial or engineered polypeptide constructs that comprise at least one antibody-derived antigen binding site.

Anti-C5 antibodies (or V_{H}/V_{L} domains derived therefrom) suitable for use herein can be generated using methods known in the art. Alternatively, art-recognized anti-C5 antibodies can be used. Antibodies that compete with any of these art-recognized antibodies for binding to C5 also can be used.

Eculizumab (also known as SOLIRIS®) is an anti-C5 antibody comprising heavy chain CDR1, CDR2 and CDR3 domains having the sequences set forth in SEQ ID NOs:1, 2 and 3, respectively, and light chain CDR1, CDR2 and CDR3 domains having the sequences set forth in SEQ ID NOs:4, 5 and 6, respectively. Eculizumab comprises a heavy chain variable region having the amino acid sequence set forth in SEQ ID NO:7 and a light chain variable region having the amino acid sequence set forth in SEQ ID NO:8. The variable regions of eculizumab are described in PCT/US1995/005688 and U.S. Pat. No. 6,355,245, the teachings of which are hereby incorporated by reference in their entirety. Eculizumab comprises a heavy chain comprising the amino acid sequence set forth in SEQ ID NO:10 and a light chain having the amino acid sequence set forth in SEQ ID NO:11. The full heavy and light chains of eculizumab are described in PCT/US2007/006606, the entire teachings of which are hereby incorporated by reference. In some embodiments, eculizumab includes a biosimilar of SOLIRIS®. As used herein, a biosimilar is a product which is highly similar (e.g., in structure, function and property) to another already approved biological medicine (e.g., a reference medicine). Representative examples of SOLIRIS® biosimilars include, e.g., monoclonal antibody ABP 959; ELIZARIA; and monoclonal antibody SB12.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®) is described in PCT/US2015/019225 and U.S. Pat. No. 9,079,949, the entire teachings of which are hereby incorporated by reference. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. The antibody can comprise, for example, the CDR1, CDR2 and CDR3 domains of the V_{H} region of ravulizumab having the sequence set forth in SEQ ID NO:12, and the CDR1, CDR2 and CDR3 domains of the V_{L} region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises V_{H} and V_{L} regions having the amino acid sequences set forth in SEQ ID NO:12 and SEQ ID NO:8, respectively.

Another exemplary anti-C5 antibody comprises heavy and light chains having the sequences shown in SEQ ID NOs:20 and 11, respectively, or antigen binding fragments and variants thereof. In other embodiments, the antibody can comprise the heavy and light chain CDRs of SEQ ID Nos:20 and 11. Accordingly, in one embodiment, the antibody comprises the CDR1, CDR2 and CDR3 domains of the V_{H} having the sequence set forth in SEQ ID NO:12, and the CDR1, CDR2 and CDR3 domains of the V_{L} region having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively.

The exact boundaries of CDRs have been defined differently according to different methods. In some embodiments, the positions of the CDRs or framework regions within a light or heavy chain variable domain can be as defined by Kabat et al. (“Sequences of Proteins of Immunological Interest.” NIH Publication No. 91-3242, U.S. Department of Health and Human Services, Bethesda, Md., 1991). In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs” (Thomas et al., Mol. Immunol., 33:1389-401, 1996).

In another embodiment, the antibody comprises V_{H} and V_{L} regions having the amino acid sequences set forth in SEQ ID NO:12 and SEQ ID NO:8, respectively. In another embodiment, the antibody comprises a heavy chain constant region as set forth in SEQ ID NO:13. In another embodiment, the antibody comprises a heavy chain polypeptide as set forth in SEQ ID NO:14 and a light chain polypeptide as set forth in SEQ ID NO:11. In another embodiment, the antibody comprises a variant human Fc region that binds to human neonatal Fc receptor (FcRn), wherein the variant human Fc CH3 region comprises Met429Leu and Asn435Ser substitutions at residues corresponding to methionine 428 and asparagine 434 of a native human IgG Fc region, each in EU numbering.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc region that binds to human neonatal Fc receptor (FcRn), wherein the variant human Fe CH3 region comprises Met429Leu and Asn435Ser substitutions at residues corresponding to methionine 428 and asparagine 434 of a native human IgG Fe region, each in EU numbering.

In another embodiments, an anti-C5 antibody described herein comprises a heavy chain CDR1 comprising, or consisting of, the following amino acid sequence: GHIFSNYWIQ (SEQ ID NO:19). In another embodiment, an anti-C5 antibody described herein comprises a heavy chain CDR2 comprising, or consisting of, the following amino acid sequence: EILPGSGHTEYTENFKD (SEQ ID NO:18).

In another embodiment, the antibody binds to human C5 at pH 7.4 and 25C with an affinity dissociation constant (K_{D}) that is in the range 0.1 nM≤K_{D}≤1 nM. In another embodiment, the antibody binds to human C5 at pH 6.0 and 25C with a K_{D}≥10 nM. In yet another embodiment, the K_{D} of the antibody or antigen-binding fragment thereof for human C5 at pH 6.0 at 25C)/(K_{D} of the antibody or antigen-binding fragment thereof for human C5 at pH 7.4 at 25C of the antibody is greater than 25.

Another exemplary anti-C5 antibody is as described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs: 21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the V_{H} region having the sequence set forth in SEQ ID NO:27, and the V_{L} region having the sequence set forth in SEQ ID NO:28.

Another exemplary anti-C5 antibody is also described in U.S. Pat. Nos. 8,241,628 and 8,883,158. In one embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the V_{H} region having the sequence set forth in SEQ ID NO:35, and the V_{L} region having the sequence set forth in SEQ ID NO:36.

Another exemplary anti-C5 antibody is described in US2016/0176954A1. In one embodiment, the antibody, or antigen binding fragment thereof, comprises heavy chain CDR1, CDR2 and CDR3 domains having the sequences set forth in SEQ ID NOs:37, 38 and 39, respectively, and light chain CDR1, CDR2 and CDR3 domains having the sequences set forth in SEQ ID NOs:40, 41 and 42, respectively. In another embodiment, the antibody comprises the V_{H} region having the sequence set forth in SEQ ID NO:43, and the V_{L} region having the sequence set forth in SEQ ID NO:44.

Another exemplary anti-C5 antibody is described in Fukuzawa T. et al. (Sci. Rep., 7:1080, 2017). In one embodiment, the antibody or antigen binding fragment thereof comprises a heavy chain comprising SEQ ID NO:45 and a light chain comprising SEQ ID NO:46.

Another exemplary anti-C5 antibody is described in US2017/0355757. In one embodiment, the antibody comprises a heavy chain variable region comprising SEQ ID NO:47 and a light chain variable region comprising SEQ ID NO:48. In another embodiment, the antibody comprises a heavy chain comprising SEQ ID NO:49 and a light chain comprising SEQ ID NO:50.

The antibodies described herein can compete for binding with, and/or bind to the same epitope on C5 as any of the above-mentioned antibodies. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, X-ray analysis of crystals of antigen:antibody complexes that provides atomic resolution of the epitope and hydrogen/deuterium exchange mass spectrometry (HDX-MS). Other methods monitor the binding of the antibody to peptide antigen fragments or variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same V_{H} and V_{L} or the same CDR1, CDR2 and CDR3 sequences are expected to bind to the same epitope.

Antibodies described herein can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti-C5 antibody described herein can, in some embodiments, comprise a variant human Fc region that binds to human neonatal Fc receptor (FcRn) with greater affinity than that of the native human Fc region from which the variant human Fc region was derived. The Fe constant region can comprise, for example, one or more (e.g., two, three, four, five, six, seven, eight or more) amino acid substitutions relative to the native human Fe region from which the variant human Fc region was derived. The substitutions can increase the binding affinity of an IgG antibody containing the variant Fe region to FcRn at pH 6.0, while maintaining the pH dependence of the interaction. Methods for testing whether one or more substitutions in the Fc region of an antibody increase the affinity of the Fc region for FcRn at pH 6.0 (while maintaining pH dependence of the interaction) are known in the art and exemplified in the working examples.

Substitutions that enhance the binding affinity of an antibody Fc region for FcRn are known in the art and include, e.g., (1) the M252Y/S254T/T256E triple substitution (Dall′Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56); and (3) the N434A or T307/E380A/N434A substitutions (Petkova, S. et al., Int. Immunol., 18:1759-69, 2006). Additional substitution pairings, for example, P257I/Q311I, P257I/N434H and D376V/N434H are described in, e.g., Datta-Mannan, A. et al. (J. Biol. Chem., 282:1709-17, 2007). The disclosures of each of these references are incorporated herein by reference in its entireties.

In some embodiments, the constant region can comprise a substitution at EU amino acid residue 255 for valine, a substitution at EU amino acid residue 309 for asparagine, a substitution at EU amino acid residue 312 for isoleucine and/or a substitution at EU amino acid residue 386.

The antibodies described herein can comprise a variant Fc region of no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions or deletions relative to the native constant region from which it was derived. In some embodiments, the variant Fc region comprises one or more amino acid substitutions selected from the group consisting of: M252Y, S254T, T256E, N434S, M428L, V259I, T250I and V308F. In some embodiments, the variant human Fc region comprises a methionine at position 428 and an asparagine at position 434, each in EU numbering. In some embodiments, the variant Fc region comprises a 428L/434S double substitution as described in, e.g., U.S. Pat. No. 8,088,376.

In some embodiments the precise location of substitutions can be shifted from the native human Fc region position as desired for antibody engineering. For example, the 428L/434S double substitution, when used in a IgG2/4 chimeric Fe, can correspond to 429L and 435S as in the M429L and N435S variants found in ravulizumab.

An antibody described herein can comprise, for example, a constant region comprising a substitution at one or more amino acid positions 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with a K_{D} that is at least 0.1 nM (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975 nM). In some embodiments, the K_{D} of the anti-C5 antibody, or antigen binding fragment thereof, is no greater than 1 nM (e.g., no greater than 0.9, 0.8, 0.7, 0.6, 0.5, 0.4, 0.3, or 0.2 nM).

In other embodiments, the K_{D} of the antibody for C5 at pH 6.0 at 25° C.)/(K_{D} of the antibody for C5 at pH 7.4 at 25C is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

An anti-C5 antibody described herein can have a serum half-life in humans that is, for example, at least 20 days (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 days). In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20% greater than the serum half-life of eculizumab (e.g., at least 30, 35, 40, 45, 50, 55, 60, 65, 70, 75, 80, 85, 90, 95, 100, 125, 150, 175, 200, 250, 300, 400 or 500% greater than the serum half-life of eculizumab).

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody.” Competing antibodies bind to the same epitope, an overlapping epitope or to adjacent epitopes (e.g., as evidenced by steric hindrance).

Anti-C5 antibodies or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are commonly immortalized by fusion with a myeloma cell (Kahler, G. & Milstein, C., Eur. J. Immunol., 6:511-9, 1976). Alternative methods of immortalization include transformation with Epstein Barr Virus, oncogenes or retroviruses, or other methods known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences that encode a monoclonal antibody or a binding fragment thereof by screening a DNA library from human B cells (Huse, W. et al., Science, 246:1275-81, 1989).

### II. Compositions

Compositions comprising an anti-C5 antibody, or antigen binding fragment thereof, as described herein, can be formulated as a pharmaceutical solution. Pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. Compositions can include, for example, a pharmaceutically acceptable salt, e.g., an acid addition salt or a base addition salt, sugars, carbohydrates, polyols and/or tonicity modifiers.

Compositions described herein can be formulated according to standard methods. Pharmaceutical formulation is a well-established art (Gennaro, “Remington: The Science and Practice of Pharmacy,” 20^{th} Edition, Lippincott, Williams & Wilkins (ISBN: 0683306472), 2000; Ansel et al., “Pharmaceutical Dosage Forms and Drug Delivery Systems,” 7^{th} Edition, Lippincott Williams & Wilkins Publishers (ISBN: 0683305727), 1999; and Kibbe, “Handbook of Pharmaceutical Excipients American Pharmaceutical Association,” 3^{rd} Edition (ISBN: 091733096X), 2000). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8° C. (e.g., 4° C.). Thus, in some embodiments, the compositions described herein are stable in storage for at least 1 year at 2-8C (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In some embodiments, the anti-C5 antibody, or antigen binding fragment thereof, is formulated as a pharmaceutical solution and administered via subcutaneous injection. Subcutaneous administration can be accomplished by means of a device.

In some embodiments, the composition comprises ravulizumab for injection. In one embodiment, the injection is a sterile, clear to translucent, slightly whitish color, preservative-free solution for intravenous use. In another embodiment, each single-dose vial contains 300 mg ravulizumab for injection at a concentration of 10 mg/mL with a pH of 7.0. In another embodiment, ravulizumab for injection requires dilution to a final concentration of 5 mg/mL. In another embodiment, each mL further comprises polysorbate 80 (0.2 mg; vegetable origin), sodium chloride (8.77 mg), sodium phosphate dibasic (1.78 mg), sodium phosphate monobasic (0.46 mg) and water.

### III. Methods of Treatment

Provided herein are methods for treating p-aHUS (including postpartum aHUS) in a human patient, comprising administering to the patient an anti-C5 antibody, or antigen binding fragment thereof (e.g., ravulizumab). As used herein, the term “subject” or “patient” is a human patient (e.g., a patient having p-aHUS).

Hemolytic uremic syndrome (HUS) is characterized by thrombocytopenia, microangiopathic hemolytic anemia, and acute renal failure. HUS is classified as one of two types: diarrheal-associated (D+ HUS; also referred to as shiga toxin producing E. coli (STEC)-HUS or typical HUS) and non-diarrheal or atypical HUS (aHUS). D+ HUS is the most common form, accounting for greater than 90% of cases and is caused by a preceding illness with a shiga-like toxin-producing bacterium, e.g., E. coli O157:H7.

aHUS can be genetic, acquired, or idiopathic. Hereditable forms of aHUS can be associated with mutations in a number of human complement components including, e.g., complement factor H (CFH), membrane cofactor protein (MCP), complement factor I (CFI), C4b-binding protein (C4BP), complement factor B (CFB), and complement component 3 (C3) (see, e.g., Caprioli et al. (2006) Blood 108:1267-1279). Certain mutations in the gene encoding CD55, though not yet implicated in aHUS, are associated with the severity of aHUS (see, e.g., Esparza-Gordillo et al. (2005) Hum Mol Genet 14:703-712). aHUS can be considered genetic when two or more (e.g., three, four, five, or six or more) members of the same family are affected by the disease at least six months apart and exposure to a common triggering agent has been excluded, or when one or more aHUS-associated gene mutations (e.g., one or more mutations in CFH, MCP/CD46, CFB, or CFI) are identified in a subject. For example, a subject can have CFH-associated aHUS, CFB-associated aHUS, CFI-associated aHUS, or MCP-associated aHUS. Up to 30% of genetic aHUS is associated with mutations in CFH, 12% with mutations in MCP, 5-10% with mutations in CFI, and less than 2% with mutations in CFB. Genetic aHUS can be multiplex (i.e., familial; two or more affected family members) or simplex (i.e., a single occurrence in a family). aHUS can be considered acquired when an underlying environmental factor (e.g., a drug, systemic disease, or viral or bacterial agents that do not result in Shiga-like exotoxins) or trigger can be identified. aHUS can be considered idiopathic when no trigger (genetic or environmental) is evident.

aHUS triggered by pregnancy (“p-aHUS”) is a rare and severe systemic disease associated with dysregulation of the alternative complement pathway that occurs in approximately 1 in 25,000 pregnancies. Hyperactivation of complement results in diffuse endothelial injury with subsequent formation of fibrin and platelet microthrombi in the vasculature, which leads to hemolysis, thrombocytopenia, and end organ dysfunction from ischemia (primarily in the form of acute kidney injury). The majority of cases of p-aHUS occur during the postpartum period and are therefore, referred to as “postpartum aHUS”. In some embodiments, the patient has severe or early onset p-aHUS. In such cases, it is necessary to prolong pregnancy in order to improve perinatal outcome.

Laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668). Blood concentrations of C3 and C4 can also be used as a measure of complement activation or dysregulation. In addition, a subject's condition can be further characterized by identifying the subject as harboring one or more mutations in a gene associated with aHUS such as CFI, CFB, CFH, or MCP (supra). Suitable methods for detecting a mutation in a gene include, e.g., DNA sequencing and nucleic acid array techniques. See, e.g., Breslin et al. (2006) Clin Am Soc Nephrol 1:88-99 and Goicoechea de Jorge et al. (2007) Proc Natl Acad Sci USA 104:240-245.

In some embodiments, the patient also has preeclampsia (PE) and/or HELLP syndrome. PE is a multi-system progressive disorder characterized by new onset hypertension and proteinuria (e.g., protein excretion of >300 mg/24 hours), or hypertension and end-organ dysfunction with or without proteinuria, after 20 weeks of gestation or postpartum in previously normotensive women. In pregnant women with PE, delivery of the fetus and placenta is the only curative treatment, which carries risks of prematurity and its associated complications. HELLP syndrome is a complication of pregnancy characterized by hemolysis, elevated liver enzymes, and a low platelet count. PE/HELLP and p-aHUS share biochemical features, wherein HELLP and p-aHUS can be part of a spectrum. Whereas aHUS is mainly characterized by microangiopathic haemolytic anaemia (MAHA) and AKI, PE/HELLP presents as kidney dysfunction, including non-cardiogenic pulmonary oedema, subcapsular hepatic haematoma and impaired liver functions in addition to AKI and MAHA (see, e.g., Elabd et al., BMJ Case Rep. 2019; 12). Generally, PE and HELLP syndrome manifest as diseases of the microvasculature and present with many of the same findings as thrombotic thrombocytopenic purpura (TTP) and hemolytic uremic syndrome (HUS). These diseases usually occur during the third trimester and necessitate delivery of the baby. Physicians recommend considering TTP and HUS in patients with suspected preeclampsia or HELLP syndrome if the TMA persists after delivery. To this end, treatment for TTP may be carried out using plasma exchange and administration with corticosteroids; and should the patient not respond, rituximab may be administered. With HUS, supportive care and cobalamin supplementation (hydroxocobalamin, folic acid and/or betaine) may be provided (Thurman et al., Clin J Am Soc Nephrol 13: 933-936, 2018).

As used herein, “gestational age” (or “menstrual age”) refers to the time elapsed between the first day of the last normal menstrual period and the day of delivery. Gestational age is conventionally expressed as completed weeks. Therefore, a 25-week, 5-day fetus is considered a 25-week fetus. The term “gestational age” is normally used instead of “menstrual age” to describe the age of the fetus or newborn infant. Gestational age is often determined by the “best obstetric estimate,” which is based on a combination of the first day of last menstrual period, physical examination of the mother, prenatal ultrasonography, and history of assisted reproduction. The best obstetric estimate is necessary because of gaps in obstetric information and the inherent variability (as great as 2 weeks) in methods of gestational age estimation. Postnatal physical examination of the infant is sometimes used as a method to determine gestational age if the best obstetric estimate seems inaccurate.

As used herein, “chronological age” (or “postnatal” age) refers to the time elapsed after birth. It is usually described in days, weeks, months, and/or years. This is different from the term “postmenstrual age.”

As used herein, “postmenstrual age” refers to the time elapsed between the first day of the last menstrual period and birth (gestational age) plus the time elapsed after birth (chronological age). Postmenstrual age is usually described in number of weeks and is most frequently applied during the perinatal period beginning after the day of birth. Therefore, a preterm infant born at a gestational age of 33 weeks who is currently 10 weeks old (chronological age) would have a postmenstrual age of 43 weeks.

As used herein, “corrected age” (or “adjusted age”) refers a term most appropriately used to describe children up to 3 years of age who were born preterm. Corrected age is calculated by subtracting the number of weeks born before 40 weeks of gestation from the chronological age. Therefore, a 24-month-old, former 28-week gestational age infant has a corrected age of 21 months according to the following equation: 24 months−[(40 weeks−28 weeks)×1 month/4 weeks]. Corrected age and chronological age are not synonymous in preterm infants. Additionally, the term “corrected age” should be used instead of “adjusted age.”

As used herein, “conceptional age” is the time elapsed between the day of conception and the day of delivery. Because assisted reproductive technologies accurately define the date of fertilization or implantation, a precise conceptional age can be determined in pregnancies resulting from such technologies.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. For example, effective treatment may refer to alleviation of one more symptoms selected from the group consisting of a reduction or cessation in severe hypertension, proteinuria, uremia, lethargy/fatigue, irritability, thrombocytopenia, microangiopathic hemolytic anemia, and renal function impairment (e.g., acute renal failure) compared to baseline.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen binding fragment thereof, clinically proven to alleviate at least one symptom of p-aHUS. An effective amount can be administered in one or more administrations.

As used herein, the phrase “end-stage renal disease” (also known as ESRD or kidney failure) refers to the last stage of chronic kidney disease, wherein the kidneys have stopped working well enough, thereby necessitating life-long dialysis or a kidney transplant for survival.

In one embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5 and 6, respectively. In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises a heavy chain variable region having the amino acid sequence set forth in SEQ ID NO: 7 and a light chain variable region having the amino acid sequence set forth in SEQ ID NO: 8. In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises a heavy chain comprising the amino acid sequence set forth in SEQ ID NO:10 and a light chain having the amino acid sequence set forth in SEQ ID NO:11.

In one embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody further comprises a variant human Fe constant region that binds to human neonatal Fe receptor (FcRn), wherein the variant human Fe CH3 constant region comprises Met-429-Leu and Asn-435-Ser substitutions at residues corresponding to methionine 428 and asparagine 434 of a native human IgG Fe constant region, each in EU numbering.

In one embodiment, the anti-C5 antibody, or antigen binding fragment is administered at a fixed dose. For example, in one embodiment, the anti-C5 antibody, or antigen binding fragment is administered at a dose of 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 40 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg, without regard to the patient's weight. In another embodiment, the anti-C5 antibody, or antigen binding fragment is administered at a sub-therapeutic dose.

In another embodiment, the anti-C5 antibody, or antigen binding fragment thereof, (e.g., ravulizumab) is administered at a dose of 2400 mg, 2700 mg, 3000 mg, 3300 mg or 3600 mg.

In another embodiment, the dose of the anti-C5 antibody, or antigen binding fragment is based on the weight of the patient. For example, in one embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody, or antigen binding fragment is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg. In another embodiment, 2400 mg or 3000 mg of the anti-C5 antibody, or antigen binding fragment thereof, (e.g., ravulizumab) is administered to a patient weighing ≥40 to <60 kg every two weeks. In another embodiment, 2400 mg or 3000 mg of the anti-C5 antibody, or antigen binding fragment thereof, (e.g., ravulizumab) is administered to a patient weighing ≥40 to <60 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg, or 11000 mg of the anti-C5 antibody, or antigen binding fragment thereof, is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg. In another embodiment, 2700 mg or 3300 mg of the anti-C5 antibody, or antigen binding fragment thereof, (e.g., ravulizumab) is administered to a patient weighing ≥60 to <100 kg every eight weeks.

In another embodiment, 10 mg, 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, 6100 mg, 6200 mg, 6300 mg, 6400 mg, 6500 mg, 6600 mg, 6700 mg, 6800 mg, 6900 mg, 7000 mg, 7100 mg, 7200 mg, 7300 mg, 7400 mg, 7500 mg, 7600 mg, 7700 mg, 7800 mg, 7900 mg, 8000 mg, 8100 mg, 8200 mg, 8300 mg, 8400 mg, 8500 mg, 8600 mg, 8700 mg, 8800 mg, 8900 mg, 9000 mg, 9100 mg, 9200 mg, 9300 mg, 9400 mg, 9500 mg, 9600 mg, 9700 mg, 9800 mg, 9900 mg, 10000 mg, 10100 mg, 10200 mg, 10300 mg, 10400 mg, 10500 mg, 10600 mg, 10700 mg, 10800 mg, 10900 mg or 11000 mg of the anti-C5 antibody, or antigen binding fragment, is administered to a patient weighing ≥100 kg. In another embodiment, 3000 mg or 3600 mg of the anti-C5 antibody, or antigen binding fragment thereof, (e.g., ravulizumab) is administered to a patient weighing ≥100 kg. In another embodiment, 3000 mg or 3600 mg of the anti-C5 antibody, or antigen binding fragment thereof, (e.g., ravulizumab) is administered to a patient weighing ≥100 kg every eight weeks.

In another embodiment, a method of treating a human patient with p-aHUS is provided, the method comprising administering to the patient an anti-C5 antibody, or antigen binding fragment thereof (e.g., ravulizumab): (a) once on Day 1 at a dose of: 2400 mg to a patient weighing ≥40 to <60 kg, 2700 mg to a patient weighing 60 to <100 kg, or 3000 mg to a patient weighing 100 kg; and (b) on Day 15 and every eight weeks thereafter at a dose of 3000 mg to a patient weighing 40 to <60 kg, 3300 mg to a patient weighing 60 to <100 kg, or 3600 mg to a patient weighing 100 kg.

In another embodiment, a method of treating a human patient with m-aHUS is provided, wherein the anti-C5 antibody, or antigen binding fragment thereof, (e.g., ravulizumab) is administered to a patient weighing 40 to <60 kg: (a) once on Day 1 at a dose of 2400 mg; and (b) on Day 15 and every eight weeks thereafter at a dose of 3000 mg.

In another embodiment, a method of treating a human patient with p-aHUS is provided, wherein the anti-C5 antibody, or antigen binding fragment thereof, (e.g., ravulizumab) is administered to a patient weighing 60 to <100 kg: (a) once on Day 1 at a dose of 2700 mg; and (b) on Day 15 of the administration cycle and every eight weeks thereafter at a dose of 3300 mg.

In another embodiment, a method of treating a human patient with p-aHUS is provided, wherein the anti-C5 antibody, or antigen binding fragment thereof, (e.g., ravulizumab) is administered to a patient weighing 100 kg: (a) once on Day 1 at a dose of 3000 mg; and (b) on Day 15 and every eight weeks thereafter at a dose of 3600 mg.

In another embodiment, a method of treating a human patient with p-aHUS is provided, the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fe region, each in EU numbering, and wherein the anti-C5 antibody, or antigen binding fragment thereof, is administered to the patient: (a) once on Day 1 at a dose of: 2400 mg to a patient weighing 40 to <60 kg, 2700 mg to a patient weighing ≥60 to <100 kg, or 3000 mg to a patient weighing ≥100 kg; and (b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In another embodiment, the anti-C5 antibody, or antigen binding fragment, is administered at a milligram per kilogram (mg/kg) dose. For example, in one embodiment, the anti-C5 antibody, or antigen binding fragment thereof, is administered at a dose of 0.1 mg/kg, 0.25 mg/kg, 0.5 mg/kg, 0.75 mg/kg, 1.0 mg/kg, 1.25 mg/kg, 1.50 mg/kg, 1.75 mg/kg, 2.0 mg/kg, 2.25 mg/kg, 2.50 mg/kg, 2.75 mg/kg, 3.0 mg/kg, 3.25 mg/kg, 3.50 mg/kg, 3.75 mg/kg, 4.0 mg/kg, 4.25 mg/kg, 4.50 mg/kg, 4.75 mg/kg, 5.0 mg/kg, 5.25 mg/kg, 5.50 mg/kg, 5.75 mg/kg, 6.0 mg/kg, 6.25 mg/kg, 6.50 mg/kg, 6.75 mg/kg, 7.0 mg/kg, 7.25 mg/kg, 7.50 mg/kg, 7.75 mg/kg, 8.0 mg/kg, 8.25 mg/kg, 8.50 mg/kg, 8.75 mg/kg, 9.0 mg/kg, 9.25 mg/kg, 9.50 mg/kg, 9.75 mg/kg, 10.0 mg/kg, 11.25 mg/kg, 11.50 mg/kg, 11.75 mg/kg, 12.0 mg/kg, 12.25 mg/kg, 12.50 mg/kg, 12.75 mg/kg, 13.0 mg/kg, 13.25 mg/kg, 13.50 mg/kg, 13.75 mg/kg, 14.0 mg/kg, 14.25 mg/kg, 14.50 mg/kg, 14.75 mg/kg, 15.0 mg/kg, 15.25 mg/kg, 15.50 mg/kg, 15.75 mg/kg, 16.0 mg/kg, 16.25 mg/kg, 16.50 mg/kg, 16.75 mg/kg, 17.0 mg/kg, 17.25 mg/kg, 17.50 mg/kg, 17.75 mg/kg, 18.0 mg/kg, 18.25 mg/kg, 18.50 mg/kg, 18.75 mg/kg, 19.0 mg/kg, 19.25 mg/kg, 19.50 mg/kg, 19.75 mg/kg, 20.0 mg/kg, 20.25 mg/kg, 20.50 mg/kg, 20.75 mg/kg, 21.0 mg/kg, 21.25 mg/kg, 21.50 mg/kg, 21.75 mg/kg, 22.0 mg/kg, 22.25 mg/kg, 22.50 mg/kg, 22.75 mg/kg, 23.0 mg/kg, 23.25 mg/kg, 23.50 mg/kg, 23.75 mg/kg, 24.0 mg/kg, 24.25 mg/kg, 24.50 mg/kg, 24.75 mg/kg or 25.0 mg/kg.

In one embodiment, the anti-C5 antibody, or antigen binding fragment is administered once per week, twice per week, three times per week, four times per week, five times per week, six times per week or daily. In another embodiment, the anti-C5 antibody, or antigen binding fragment, is administered twice daily. In another embodiment, anti-C5 antibody, or antigen binding fragment, is administered once every two weeks, once every three weeks, once every four weeks, once every five weeks, once every six weeks, once every seven weeks, once every eight weeks, once every nine weeks, once every ten weeks, once every eleven weeks or once every twelve weeks. In another embodiment, the anti-C5 antibody, or antigen binding fragment, is administered at a loading dose on Day 1, followed by a different maintenance dose on Day 15 and every eight weeks thereafter.

In other embodiments, the anti-C5 antibody, or antigen binding fragment thereof, is administered immediately after symptoms first occur. In other embodiments, treatment begins 1-20 days after symptoms first occur. In other embodiments, treatment begins 1, 2, 3, 4, 5, 6, 7, 8, 9, 10, 11, 12, 13, 14, 15, 16, 17, 18, 19, or 20 days after symptoms first occur. In other embodiments, treatment begins 5-15 days after delivery. In other embodiments, treatment begins 1, 2, 3, 4, 5, 6, 7, 8, 9, 10, 11, 12, 13, 14, 15, 16, 17, 18, 19, or 20 days after delivery. In another embodiment, the anti-C5 antibody, or antigen binding fragment thereof, is administered for one or more administration cycles. In one embodiment, the administration cycle is 26 weeks. In another embodiment, the treatment comprises at least 1, 2, 3, 4, 5, 6, 7, 8, 9, 10 or 11 cycles. In another embodiment, the patient is treated for about 1, 2, 3, 4, 5, or 6 months. In another embodiment, the treatment continues for the lifetime of the human patient.

In some embodiments, the patient has not previously been treated with a complement inhibitor (e.g., the patient is a complement inhibitor treatment-naïve patient). In another embodiment, the patient is a complement inhibitor treatment-naïve patient who has previously received plasma exchange and/or dialysis.

The anti-C5 antibody, or antigen binding fragment, can be administered via any suitable means. In one embodiment, the anti-C5 antibody, or antigen binding fragment, is administered intravenously. In another embodiment, the anti-C5 antibody, or antigen binding fragment, is administered subcutaneously.

In some embodiments, the patients treated according to the methods described herein have been vaccinated against meningococcal infections within three years prior to, or at the time of, initiating treatment. In one embodiment, patients who received treatment less than two weeks after receiving a meningococcal vaccine are also treated with appropriate prophylactic antibiotics until two weeks after vaccination. In another embodiment, patients treated according to the methods described herein are vaccinated against meningococcal serotypes A, C, Y, W135 and/or B.

In another aspect, the treatment regimens described are sufficient to maintain particular serum trough concentrations of the anti-C5 antibody, or antigen binding fragment thereof. The treatment can maintain, for example, a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment maintains a serum trough concentration of the anti-C5 antibody, or antigen binding fragment thereof, of 100 μg/mL or greater. In another embodiment, the treatment maintains a serum trough concentration of the anti-C5 antibody, or antigen binding fragment thereof, of 150 μg/mL or greater. In another embodiment, the treatment maintains a serum trough concentration of the anti-C5 antibody, or antigen binding fragment thereof, of 200 μg/mL or greater. In another embodiment, the treatment maintains a serum trough concentration of the anti-C5 antibody, or antigen binding fragment thereof, of 250 μg/mL or greater. In another embodiment, the treatment maintains a serum trough concentration of the anti-C5 antibody, or antigen binding fragment thereof, of 300 μg/mL or greater. In another embodiment, the treatment maintains a serum trough concentration of the anti-C5 antibody, or antigen binding fragment thereof, of between 100 μg/mL and 200 μg/mL. In another embodiment, the treatment maintains a serum trough concentration of the anti-C5 antibody, or antigen binding fragment thereof, of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 g 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 g 240 μg, 245 μg, 250 μg, 255 μg or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. The anti-C5 antibody can be administered, for example, to the patient in an amount and with a frequency to maintain a free C5 concentration of 0.2 μg/mL, 0.3 μg/mL, 0.4 μg/mL, 0.5 μg/mL or below. In another embodiment, the treatment described herein reduces free C5 concentration by greater than 99% throughout the treatment period. In another embodiment, the treatment reduces free C5 concentration greater than 99.5% throughout the treatment period.

In another aspect, the methods of treating p-aHUS described herein can be used alone or in combination with one more additional therapies and/or therapeutic agents. For example, in one embodiment, the method further comprises administering to the patient an anti-inflammatory agent (e.g., prednisone).

### IV. Outcomes

Provided herein are methods for treating p-aHUS in a patient comprising administering to the patient an anti-C5 antibody, or antigen binding fragment thereof (e.g., ravulizumab).

Symptoms of p-aHUS include, but are not limited to, severe hypertension, proteinuria, uremia, lethargy/fatigue, irritability, thrombocytopenia, microangiopathic hemolytic anemia, and renal function impairment (e.g., acute renal failure). Patients treated according to the methods disclosed herein preferably experience improvement in at least one sign of p-aHUS.

In other embodiments, the treatment results in terminal complement inhibition.

In other embodiments, the treatment produces a shift toward normal levels of a hemolysis-related hematologic biomarker selected from the group consisting of free hemoglobin, haptoglobin, reticulocyte count, PNH red blood cell (RBC) clone and D-dimer.

In another embodiment, the treatment produces an increase in hemoglobin stabilization from the patient's pre-treatment baseline. In another embodiment, the treatment results in a ≥20 g/L increase in hemoglobin.

In other embodiments, the treatment results in platelet normalization (150×10 IL). In other embodiments, the treatment results in platelet normalization (≥150×10⁹/L) for at least 28 days (e.g., at least 28 days, 1 month, 2 months, 3 months, 4 months, 5 months, 6 months, 7 months, 8 months, 9 months, 10 months, 11 months, 1 year, or two years). In other embodiments, the treatment results in platelet normalization 5-12 days after initiating treatment. For example, in one embodiment, platelet normalization occurs 5, 6, 7, 8, 9, 10, 11, or 12 days after initiating treatment. In a particular embodiment, platelet normalization occurs 8 days after initiating treatment.

In other embodiments, the treatment results in LDH normalization (246 U/L). In other embodiments, the treatment results in LDH normalization (<246 U/L) for at least 28 days (e.g., at least 28 days, 1 month, 2 months, 3 months, 4 months, 5 months, 6 months, 7 months, 8 months, 9 months, 10 months, 11 months, 1 year, or two years).

In other embodiments, the treatment results in LDH normalization 5-12 days after initiating treatment. For example, in one embodiment, LDH normalization occurs 5, 6, 7, 8, 9, 10, 11, or 12 days after initiating treatment. In another embodiment, LDH normalization occurs 8 days after initiating treatment. In another embodiment, LDH and platelet normalization occurs 8 days after initiating treatment.

In other embodiments, the treatment results in a ≥25% improvement from baseline in serum creatinine. In other embodiments, the treatment results in a ≥25% improvement from baseline in serum creatinine at two separate assessments ≥28 days apart. In other embodiments, the treatment results in a ≥25% improvement from baseline in serum creatinine for at least 28 days (e.g., at least 28 days, 1 month, 2 months, 3 months, 4 months, 5 months, 6 months, 7 months, 8 months, 9 months, 10 months, 11 months, 1 year, or two years).

In other embodiments, the treatment results in a complete TMA response (i.e., platelet normalization (≥150×10⁹/L), LDH normalization (<246 U/L), and a ≥25% improvement from baseline in serum creatinine). In other embodiments, the treatment results in a complete TMA response for at least 28 days (e.g., at least 28 days, 1 month, 2 months, 3 months, 4 months, 5 months, 6 months, 7 months, 8 months, 9 months, 10 months, 11 months, 1 year, or two years). In other embodiments, the treatment confers complete TMA response in the patient in fewer than about 57 days (e.g., 56, 55, 54, 53, 52, 51, 50, 49, 48, 48, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 day(s)). In other embodiments, the treatment confers complete TMA response in the patient in fewer than about 43 days. In other embodiments, the treatment confers complete TMA response in the patient in fewer than about 22 days. In other embodiments, the treatment confers complete TMA response in the patient in fewer than about 15 days. In other embodiments, treatment of p-aHUS patients with the anti-C5 antibody, or antigen binding fragment thereof, (e.g., ravulizumab) confers complete TMA response in about 32 days (range: 8 days to 57 days, with 95% CI between 9 days and 46 days).

In other embodiments, the treatment results in a modified complete TMA Response (i.e., platelet normalization (≥150×10⁹/L), LDH normalization (≤246 U/L), and the patient is off dialysis if they were on dialysis at baseline or a ≥25% improvement from baseline in serum creatinine for a patient who was off dialysis at baseline). In other embodiments, the treatment results in a modified complete TMA Response for at least 28 days (e.g., at least 28 days, 1 month, 2 months, 3 months, 4 months, 5 months, 6 months, 7 months, 8 months, 9 months, 10 months, 11 months, 1 year, or two years).

In other embodiments, the treatment results in hematological normalization comprising platelet count normalization (e.g., ≥150×109/L), lactate dehydrogenase (LDH) normalization (e.g., ≤246 U/L), preferably both platelet count and LDH normalization, optionally together with improvement in serum creatinine (e.g., ≥25% improvement) over time (e.g., 2 separate assessments ≥28 days apart).

In other embodiments, the treatment produces a reduction in the need for blood transfusions. In another embodiment, the treatment produces a greater than 70% increase in transfusion avoidance.

In other embodiments, the treatment improves dialysis requirement status compared to baseline, e.g., a reduction or total discontinuation of dialysis. In one embodiment, the treatment results in a discontinuation of dialysis within 15 to 30 days. For example, in other embodiments, dialysis is discontinued within 15, 16, 17, 18, 19, 20, 21, 22, 23, 24, 25, 26, 27, 28, 29, or 30 days after initiating treatment. In one embodiment, dialysis is discontinued within about 21 days after initiating treatment.

In other embodiments, the treatment produces a reduction in major adverse vascular events (MAVEs).

In other embodiments, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale. In one embodiment, the treatment produces a change from baseline in quality of life as assessed via the FACIT-Fatigue Scale by one or more (e.g., 1, 2, or 3) points. In another embodiment, the treatment produces a change from baseline in quality of life as assessed via the FACIT-Fatigue Scale by 3 points, 150 days or more (e.g., 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 205 days, 210 days, 215 days, 220 days, or 225 days) after initiating treatment.

In other embodiments, the treatment results in a shift towards normal levels of eGFR (e.g., ≥90). In another embodiment, the treatment improves eGFR status compared to baseline. In other embodiments, the treatment improves eGFR status compared to baseline within 5-10 days of initiating treatment. For example, in one embodiment, the treatment improves eGFR status compared to baseline within 5, 6, 7, 8, 9, or 10 days of initiating treatment. In another embodiment, the treatment improves eGFR status compared to baseline within 8 days of initiating treatment.

In other embodiments, the treatment prolongs pregnancy (e.g., by days, weeks, or months). For example, in some embodiments, the treatment prolongs pregnancy by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In other embodiments, the treatment prolongs pregnancy by 1, 2, 3, 4, 5, 6, 7, 8, 9, 10, 11, 12, 13, 14, 15, 16, 17, 18, 19, or 20 weeks. In other embodiments, the treatment prolongs pregnancy by 1, 2, 3, 4, or 5 months. In other embodiments, the treatment advances gestational age (e.g., by days, weeks, or months). For example, in some embodiments, the treatment advances gestational age by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In other embodiments, the treatment advances gestational age by 1, 2, 3, 4, 5, 6, 7, 8, 9, 10, 11, 12, 13, 14, 15, 16, 17, 18, 19, or 20 weeks. In other embodiments, the treatment advances gestational age by 1, 2, 3, 4, or 5 months.

In other embodiments, the treatment prevents end-stage renal disease (ESRD). In other embodiments, the treatment prolongs time to ESRD (e.g., by days, weeks, months, or years), for example, by at least 10 months, at least 20 months, at least 40 months, at least 60 months, at least 80 months, at least 100 months, or at least 120 months. In some embodiments, the treatment prolongs time to ESRD after initial manifestation of thrombotic microangiopathies (TMA). TMA manifestations can be analyzed using routine methods, such as, (A) occurrence of a change in ≥two laboratory values selected from (1) platelet count decrease ≥25% and <lower limit of normal (LLN); (2) increase in serum creatinine (SCr) ≥25% and >upper limit of normal (ULN) and (3) increase in LDH ≥25% and >ULN; (B) a clinical sign/symptom selected from (1) thrombosis; (2) seizure; (3) reduction in renal function; (4) proteinuria; (5) hematuria; (6) increased hemolytic anemia; (7) biopsy-proven TMA; or (8) extrarenal signs of TMA (e.g., confusion, cardiovascular abnormalities, pericarditis, gastrointestinal symptoms/diarrhea); or (C) compensatory requirements such as plasma exchange/plasma infusion (PE/PI); dialysis; blood transfusions; or renal transplant.

In other embodiments, the treatment prolongs survival of the patient (e.g., by days, weeks, months, or years).

### V. Kits and Unit Dosage Forms

Also provided herein are kits that include a pharmaceutical composition containing an anti-C5 antibody, or antigen binding fragment thereof (e.g., any of those described herein previously) and a pharmaceutically acceptable carrier, in a therapeutically effective amount adapted for use in the methods described herein. The kits optionally also can include instructions, e.g., comprising administration schedules, to allow a practitioner (e.g., a physician, nurse or patient) to administer the composition contained therein to administer the composition to a patient having p-aHUS. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody, or antigen binding fragment thereof.

In one embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5 and 6, respectively; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises a heavy chain variable region having the amino acid sequence set forth in SEQ ID NO: 7 and a light chain variable region having the amino acid sequence set forth in SEQ ID NO: 8; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises a heavy chain comprising the amino acid sequence set forth in SEQ ID NO:10 and a light chain having the amino acid sequence set forth in SEQ ID NO:11; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of eculizumab and (b) instructions for using eculizumab in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fc region, each in EU numbering; and (b) instructions for using the anti-C5 antibody, or antigen binding fragment thereof, in the methods described herein.

In another embodiment, a kit for treating p-aHUS (e.g., postpartum aHUS) in a human patient is provided, the kit comprising: (a) a dose of ravulizumab and (b) instructions for using ravulizumab in the methods described herein.

Unless otherwise defined, all technical and scientific terms used herein have the same meaning as commonly understood by one of ordinary skill in the art to which the present disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries (e.g., PUBMED, NCBI or UNIPROT accession numbers), and other references mentioned herein are incorporated by reference in their entirety.

### Example

### Example 1: Efficacy and Safety of Ravulizumab in Patients with Postpartum Atypical Haemolytic Uraemic Syndrome

A. Trial Oversight and Study Design

The 311 clinical trial (NCT02949128) is a phase 3, single arm, multicenter study designed to evaluate the efficacy and safety of ULTOMIRIS® (ravulizumab) administered by intravenous (IV) infusion to adults with aHUS naïve to complement inhibitor treatment. Patients included in this analysis were ≥18 years of age and weighed ≥40 kg with active TMA (thrombocytopenia, evidence of hemolysis and kidney dysfunction) at postpartum and evidence of TMA lasting for ≥3 days after delivery. Patients could meet the platelet and lactate dehydrogenase (LDH) criteria (<150×10⁹ and ≥1.5× upper limit of normal, respectively) based on results from local laboratories, but the serum creatinine criteria (≥upper limit of normal) must have been confirmed by the central laboratory at baseline.

The study consisted of an initial evaluation period of 183 days. ULTOMIRIS® (ravulizumab) was administered to the postpartum-aHUS patients based on the patient's body weight at an initial loading dose and then two weeks later at a maintenance dose at once every 8-week intervals as set forth in Table 1. Specifically, ULTOMIRIS® (ravulizumab) was administered via an intravenous (IV) loading dose of 2400 mg, 2700 mg and 3000 mg in patients ≥40-<60 kg, ≥60-<100 kg, and >100 kg, respectively, on Day 1. Maintenance doses of 3000 mg, 3300 mg, 3600 mg, respectively, were administered on Day 15 and then every 8 weeks thereafter. Baseline was defined as the period of screening up to before the point of the first study drug infusion, including Day 1.

TABLE 1
Ravulizumab Weight Based Dosage Regimen

| Body Weight | Loading | Maintenance | Dosing |
|---|---|---|---|
| Range (kg) | Dose (mg) | Dose (mg) | interval |
| ≥40 kg to < 60 kg | 2,400 | 3,000 | Every 8 weeks |
| ≥60 kg to < 100 kg | 2,700 | 3,300 | Every 8 weeks |
| >100 kg | 3,000 | 3,600 | Every 8 weeks |

Patients must have received meningococcal vaccination according to local and national guidelines at the time of commencing therapy and were also required to receive antibiotic prophylaxis from the time of first dose of ULTOMIRIS® (ravulizumab) until at least 2 weeks after vaccination.

Patients with ADAMTS13 deficiency (activity <5%); Shiga toxin-producing Escherichia coli-HUS; hematopoietic stem cell transplantation in the 6 months prior to screening and history of malignancy within 5 years of screening were excluded. Patients who had received complement inhibitors, immunosuppressive therapies (except for kidney transplant regimens), steroids, patients who received tranexamic acid within 7 days, and patients on chronic dialysis were also excluded. Plasma exchange/infusion (PE/PI) was allowed up to, but not after, the first dose of ULTOMIRIS® (ravulizumab), but patients were excluded if therapy exceeded 28 days.

The protocol was approved by the Institutional Review Board or Independent Ethics Committee at each participating center, and the study was conducted in accordance with the Declaration of Helsinki and the Council for International Organizations of Medical Sciences International Ethical Guidelines.

B. Efficacy and Safety Endpoints

The primary efficacy endpoint was complete TMA response through an initial evaluation period of 183 days. The criteria for complete TMA response were platelet count normalization (≥150×10⁹/L), LDH normalization (≤246 U/L) and ≥25% improvement in serum creatinine from baseline met concurrently at two separate assessments ≥28 days apart, and any measurement in between. When a patient was on dialysis at baseline, the first valid baseline value was the first assessment at 6 or more days post-dialysis. Patients were considered as being on dialysis at baseline if dialysis occurred within 5 days prior to ULTOMIRIS® (ravulizumab) initiation.

Secondary objectives of the study included: time to complete TMA response; change in hematologic variables (platelets, LDH, and hemoglobin); change in estimated glomerular filtration rate (eGFR) values; and dialysis requirement status. Exploratory genetic analysis by whole exome sequencing was conducted on patients in the original study. Additional genetic analysis performed at the centers treating the individual patients included in this analysis were also included. Safety and tolerability of ULTOMIRIS® (ravulizumab) were evaluated by clinical and laboratory assessment and frequency of adverse events (AEs) and serious AEs (SAE).

C. Results

i. Patient Characteristics

Eight postpartum patients with a median age of 37.7 (range; 22.1-45.2) were identified and diagnosed within 12 days of delivery. These patients met the inclusion criteria, were enrolled, and received ≥1 dose of ULTOMIRIS® (ravulizumab). Seven patients were of white ethnicity (one was Asian). Six patients (75%) had received plasma exchange before ULTOMIRIS® (ravulizumab) therapy. Five patients (63%) were on dialysis before ULTOMIRIS® (ravulizumab) therapy. None of the patients reported breastfeeding during the study. Baseline demographics and disease characteristics are shown in Table 2.

At baseline, and prior to any ULTOMIRIS® (ravulizumab) dose, all patients presented with acute, severe medical emergency associated with the pregnancy or delivery, including intrauterine death, hemorrhage, and the need for blood transfusions. All patients had complicated deliveries and presented postpartum. Five out of eight patients had urgent deliveries of which 4 were cesarean sections with severe complications postoperatively. A fifth patient had a planned cesarean section which also had severe complications after delivery. Pre-eclampsia and hypertension were reported in two patients each, and renal failure and gestational diabetes in one patient each. Two patients suffered placental abruption, resulting in antenatal fetal death in 1 of these cases. Five patients underwent emergency cesarean section with complications occurring in four cases, hemorrhage in two, secondary hysterectomy in two (one with hemorrhage) and fetal death in one case. Additional data on the events prior to TMA are detailed in Table 3.

All patients completed the 183-day initial evaluation period with no study or drug discontinuations.

TABLE 2
Baseline Demographics and Disease Characteristics

| Age at time of first infusion (years) category | |
|---|---|
| 18 to < 30 years | 2 (25.0) |
| 30 to < 40 years | 3 (37.5) |
| 40 to < 50 years | 3 (37.5) |
| Race | |
| Asian | 1 (12.5) |
| White | 7 (87.5) |
| ADAMTS13 activity > 5% | 8 (100) |
| Extrarenal signs or symptoms of aHUS | 6 (75) |
| prior to first infusion of drug | |
| Baseline laboratory values, | |
| median (min, max) ^{a} | |
| Platelet count, ×10⁹/L | 119 (36, 473) |
| LDH, U/L | 576 (280, 876) |
| Serum creatinine, μmol/L | 408 (51, 758) |
| HGB, g/L | 72.8 (63, 105.5) |
| eGFR, mL/min/1.73 m^{2(b)} | 10.0 (10, 18) |
| Median (min, max) time between first | 6 (2, 18) |
| symptoms of aHUS and first dose, days | |
| Median (min, max) time from delivery | 11 (5, 19) |
| to first dose, days ICU care required | 7 (87.5) |
| Median (min, max) stay in ICU, days | 9 (2, 21) |
| Received PE/PI related to this TMA prior | 6 (75) |
| to first infusion of drug | |
| On dialysis within 5 days of first dose | 5 (62.5) |
| Median (min, max) time on dialysis | 5 (4, 8) |
| prior to first dose, days | |
| Patients with ≥ 1 identified pathogenic | 2 (25) |
| variant or autoantibody | |
| CFB | 1 (12.5) |
| Anti-CFH antibodies | 1 (12.5) |
| None | 5 (65.5) |
| Data not available | 1 (12.5) |

^{a} Baseline values may be after PE/PI in some patients.
^{b}eGFR in patients on dialysis was set to 10 mL/min/1.73 m², and eGFR was calculated using the Modification of Diet in Renal Disease formula.
Data displayed as n (%) unless otherwise stated.
ADAMTS13 a disintegrin and metalloproteinase with a thrombospondin type 1 motif, member 13,
aHUS atypical hemolytic uremic syndrome,
CFB complement factor B,
CFH complement factor H,
eGFR estimated glomerular filtration rate,
HGB hemoglobin,
ICU intensive care unit,
LDH lactate dehydrogenase,
PE plasma exchange,
PI plasma infusion,
TMA thrombotic microangiopathy.

TABLE 3
Individual Patient Clinical Profile

| Patient | 1 | 2 | 3 | 4 | 5 | 6 | 7 | 8 |
|---|---|---|---|---|---|---|---|---|
| Age | 39 | 22 | 42 | 31 | 45 | 28 | 42 | 36 |
| Prior | 1 | 0 | IVF multiple | 0 | 0 | 3 | 3 | 0 |
| pregnancies | | | courses | | | | | |
| Genetic data | CFB | CFH | No | No | CFHR1/ | Unknown | Heterozygous | No |
| | pathogenic | antibodies. | pathogenic | pathogenic | CFHR3 | | CFHR1/ | pathogenic |
| | variant. | Analysis | variants. | variants. | heterozygous | | CFHR3 | variants. |
| | Analysis | centrally | Analysis by | Analysis by | deletion. | | deletion. | Analysis |
| | centrally | by whole | individual | individual | Analysis by | | Analysis by | by |
| | by whole | exome | center | center | individual | | individual | individual |
| | exome | sequencing | | | center | | center | center |
| | sequencing | and by | | | | | | |
| | | individual | | | | | | |
| | | center | | | | | | |
| Clinical | Prior | New onset | IVF with | Renal | Pre- | Prior | Gestational | Pre- |
| observations | hypertension | hypertension | twin | failure and | eclampsia, | anemia, | diabetes, | eclampsia |
| prior to TMA | and on | during | pregnancy. | on | new-onset | oliguria | placenta | |
| Presentation | antihypertensives | pregnancy, | Placental | antihypertensives | insulin | 2-3 days | previa | |
| and delivery | | stagnation | abruption | | dependent | prior, | | |
| | | of growth | | | diabetes, | antenatal | | |
| | | and | | | arterial | fetal | | |
| | | suspicious | | | hypertension, | death | | |
| | | fetal | | | elevated | | | |
| | | heartbeat | | | ALT, AST | | | |
| | | CTG | | | | | | |
| Time, | 1 | 6 | 12 | 1 | 1 | 0 | 0 | 5 |
| delivery to | | | | | | | | |
| diagnosis | | | | | | | | |
| (days) | | | | | | | | |
| Delivery | Vaginal | Urgent | Urgency | Vaginal | Urgent | Urgent | Planned | Urgent |
| notes | delivery, | C-section | C-section due | delivery | C-section | C-section | C-section | vaginal |
| | TMA | due to | to placental | with no | due to | due to | caused | delivery |
| | symptoms | suspicious | abruption; | complications; | preeclamp | fetal | bleeding, | due to |
| | day 1 | fetal | massive | first | sia; | death, | resulting in | pre- |
| | postpartum | heartbeat, | bleeding | TMA | intraabdo | complete | patient | eclampsia |
| | initially | bleeding, | complications | symptoms | minal | placental | needing | |
| | managed | hemorrhagic | post | on day of | hemorrhage, | abruption | multiple | |
| | with PI | shock | hysterectomy, | delivery | abruptio | | transfusions | |
| | and RBC | | Initial | | placentae, | | and | |
| | transfusions | | TMA | | HELLP, | | hysterectomy | |
| | | | manifestations | | hysterectomy | | | |
| | | | 3 days post | | 2 days after | | | |
| | | | delivery | | cesarean | | | |
| | | | | | section | | | |
| Gestational | 34 | >37 | 23 | 40 | 35 | 36-37 | 35 | 39 |
| age at | | | | | | | | |
| delivery | | | | | | | | |
| (weeks) | | | | | | | | |
| BP at TMA | 153/96 | 165-200/ | 120-160/70 | 160/100 | 160/100 | Normal | 163/88 | 179/90 |
| onset | | 80-100 | | | | BP noted | | |
| (mmHg)^{a} | | | | | | | | |
| Baby | Live birth | Live birth | Premature | Live birth | Live birth | Prenatal | Live birth | Live birth |
| outcome | | | twins; 1 died | | | death | | |
| | | | immediately | | | | | |
| | | | (brain | | | | | |
| | | | halves had | | | | | |
| | | | not separated) | | | | | |
| Dialysis at | No | Started | Started day | No | Started on | 1 day on | Started on | No |
| baseline | | day −8 | −5 until day | | day −7 | day −4 | day −5 until | |
| | | until day | +9 | | until day −5 | | day +21 | |
| | | +3 | | | | | | |
| Dialysis at | No | No | No | No | No | No | No | No |
| Day 183 | | | | | | | | |
| Baseline | 18 | 10 | 10 | 29 | 10 | 10 | 10 | 12 |
| eGFR | | | | | | | | |
| (mL/min/1.7 | | | | | | | | |
| 3 m²)^{b} | | | | | | | | |
| eGFR at Day | 108 | 92 | 27 | 81 | 104 | 99 | 17 | 104 |
| 183 | | | | | | | | |
| (mL/min/1.7 | | | | | | | | |
| 3 m)^{b} | | | | | | | | |
| Baseline | 210 | 257 | 619 | 469 | 104 | 269 | 361 | 347 |
| serum | | | | | | | | |
| creatinine | | | | | | | | |
| (μmol/L) | | | | | | | | |
| Serum | 54 | 62 | 181 | 77 | 55 | 62 | 220 | 57 |
| creatine at | | | | | | | | |
| Day 183 | | | | | | | | |
| (μmol/L) | | | | | | | | |

^{a}Blood pressure data were provided by indivic ual treatment centers.
^{b}eGFR was calculated using the Modification of Diet in Renal Disease formula;
dialysis eGFR was set as an eGFR of 10 ml/min ALT alanine aminotransferase, AST aspartate iminotransferase, BP blood pressure, CFB complement factor B, CFH complement factor H, CFHR1 complement factor H-related protein 1, CFHR3 complement factor H-related protein 3, CTG cardiotocography, eGFR estimated glomerular filtration rate, HELLP hemolysis, elevated liver enzymes and low platelets, IVF in-vitro fertilization, PI plasma infusion, RBC red blood cell, TMA thrombotic microangiopathy Baseline was defined as the period of screening up to before the point of the first study drug infusion, including Day 1.

ii. Primary Endpoint

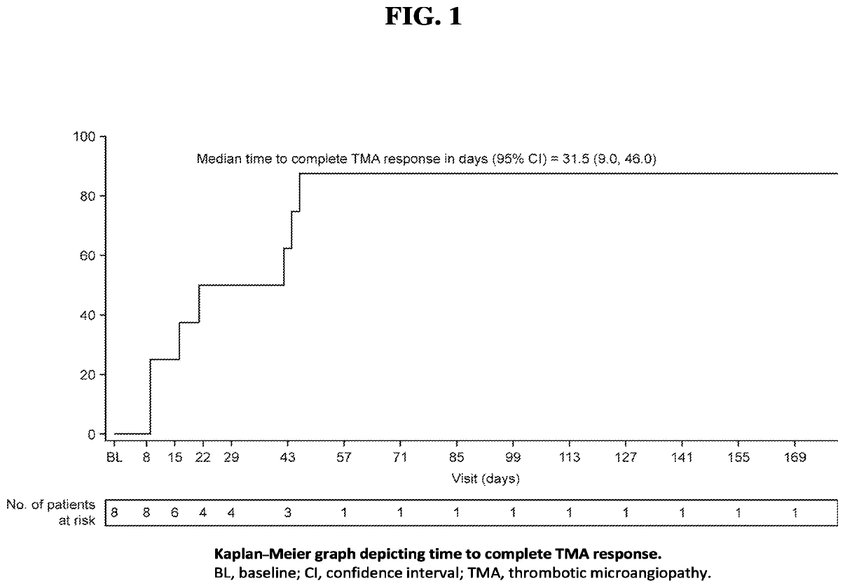

During the initial evaluation period, 7 of 8 patients (87.5%) met the primary endpoint of complete TMA response (FIG. 1). The patient that did not achieve complete TMA response had a rapid response to ULTOMIRIS® (ravulizumab) treatment, with normalization of both platelets and LDH on Day 8. She had a dialysis session 5 days before first dose, and baseline creatinine was the value obtained on Day 8 (≥6 days after last dialysis session as defined by protocol). On Day 8, this patient already had improved serum creatinine levels to 51 μmol/L and an additional improvement of 25% in serum creatinine from this value was not reached, which would have been the requirement to meet complete TMA response criteria.

iii. Secondary Endpoints

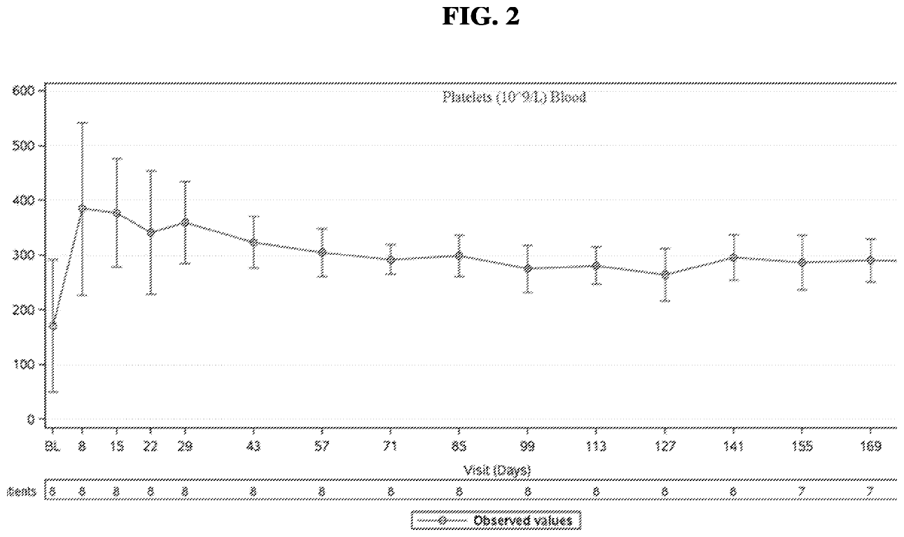

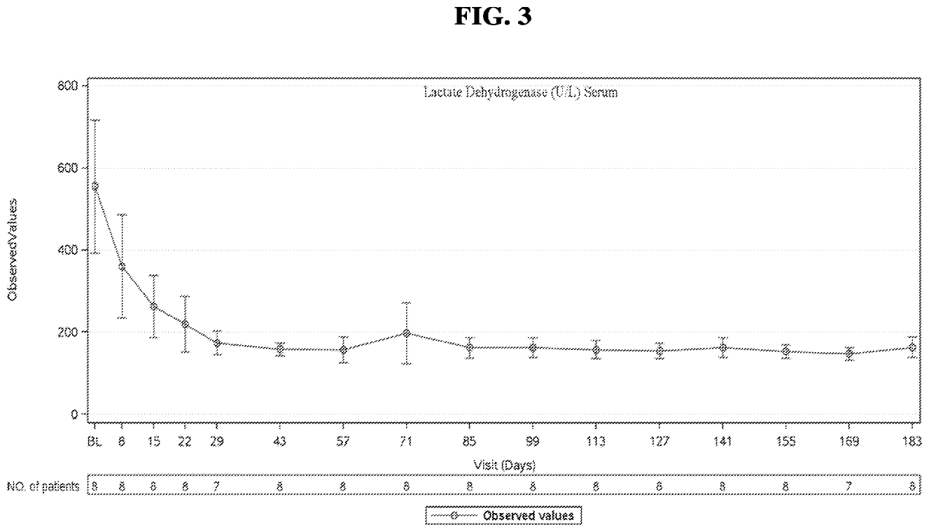

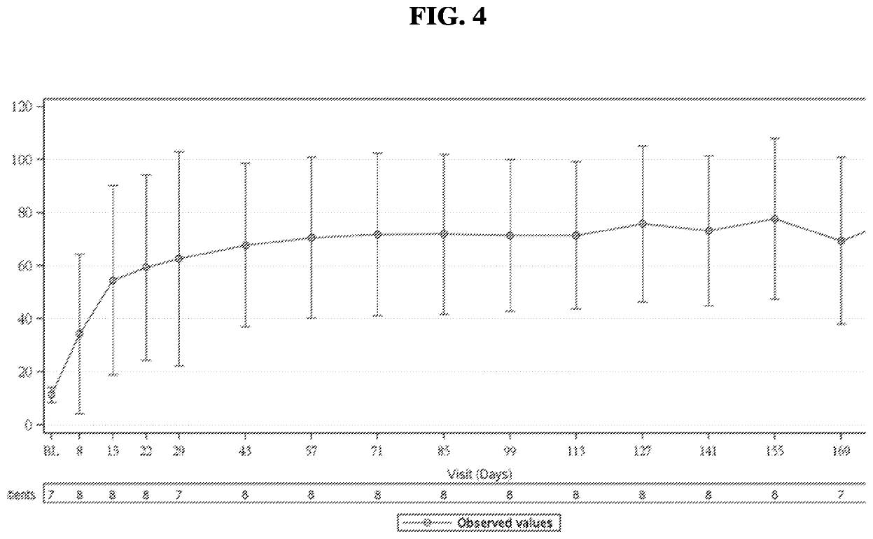

The median (95% CI) time to complete TMA response was 31.5 (9.0, 46.0) days (FIG. 1). Hematologic normalization, platelet count and LDH normalization were observed in all patients (100%). Platelet count and LDH values both rapidly improved (FIG. 2 and FIG. 3). Overall, eGFR showed rapid improvement at 8 days, which continued to improve throughout the initial evaluation period (FIG. 4). All patients on dialysis at baseline were able to discontinue dialysis within 21 days of commencing treatment with ULTOMIRIS® (ravulizumab). By Day 183, all five patients on dialysis at baseline had discontinued dialysis, and all eight patients improved eGFR status from baseline to Day 183.

iv. Safety

Adverse events post-treatment initiation were recorded in all eight patients included in the analysis (see Table 4). The most common adverse events reported in at least two patients were headache and pyrexia (occurring in three patients each). Other adverse events reported in two patients each included constipation, urinary tract infection, nasopharyngitis, alopecia, hypertension, arthralgia, increased alanine aminotransferase, and increased aspartate aminotransferase. Three possible non-severe treatment-related adverse events (as determined by investigator) were noted in two patients (arthralgia and nasopharyngitis; urinary tract infection). Both patients recovered from these events. One serious adverse event was reported. This event was a routine renal biopsy unrelated to treatment with ULTOMIRIS® (ravulizumab). No deaths or meningococcal infections occurred.

TABLE 4
Summary of Adverse Events

| | | |
|---|---|---|
| Any AE | 8 (100.0) | 71 |
| Any SAE | 1 (12.5) | 1 |
| Fatal TEAEs | 0 (0.0) | 0 |
| TEAEs or TESAEs resulting in | 0 (0.0) | 0 |
| drug discontinuation | | |
| TEAEs or TESAEs resulting in | 0 (0.0) | 0 |
| study discontinuation | | |
| Meningococcal infections | 0 (0.0) | 0 |
| Treatment-related AEs (all | 2 (25.0) | 3 |
| considered possibly-related) | | |

AE adverse event,
SAE serious adverse event,
TEAE treatment-emergent adverse event,
TESAE treatment-emergent serious adverse event.

D. Discussion

This is the largest prospective interventional study to evaluate the efficacy and safety of a C5 inhibitor in patients with aHUS presenting postpartum. All patients analyzed presented immediately postpartum with severe medical emergencies and complications after delivery. Patients responded rapidly to treatment with ULTOMIRIS® (ravulizumab), with 7/8 (84.5%) patients reaching the primary endpoint of complete TMA response by Day 43 (median time 31.5 days). No safety concerns were identified.

The data obtained in this study show that a higher proportion of patients presenting postpartum resolved TMA with ULTOMIRIS® (ravulizumab) treatment compared to the full cohort of patients with aHUS in the 311 study. Patients in this analysis received treatment soon after the first symptoms (range; 2-18 days), whereas the time to treatment in the 311 study was broader, with patients receiving their first dose of ULTOMIRIS® (ravulizumab) as early as at the onset of symptoms or as late as 215 months after the first symptom of aHUS, highlighting the importance of early treatment. Moreover, the median time to complete TMA response in this subgroup of patients was rapid at 31.5 days (38.2 days for patients on dialysis) versus 86 days for the full cohort in the 311 study. Previous clinical trial data have demonstrated that renal outcomes are better in patients initiating complement inhibitor treatment within 7 days of disease manifestation than in patients initiating treatment after 7 days. Although by the criteria of the study one patient did not achieve a complete TMA response, this patient responded to treatment with ULTOMIRIS® (ravulizumab) and improved in all clinical parameters, including serum creatinine levels, normalization of platelets and LDH by Day 8, and complete recovery of renal function at last follow-up.

As previously noted, patients presented in a severe condition. 62.5% of patients required dialysis at baseline and 87.5% of patients required intensive care unit-level care, similar to other reports detailing patients with aHUS triggered by pregnancy.

Regarding pathogenic variants, no association was found between the identification of a complement abnormality and response to ULTOMIRIS® (ravulizumab) (one patient had a complement factor B (CFB) variant and one had anti-complement factor H (CFH) antibodies). The subgroup of patients analyzed here had complicated deliveries with significant bleeding, which could have triggered the syndrome, as suggested in other studies. One patient with a severe predisposition (CFB pathogenic variant) had a normal delivery, whereas some of the patients with no identified pathogenic variants had severe bleeding complications, hypertension and pre-eclampsia, and consequently a more severe clinical presentation. In the present study, five patients underwent emergency deliveries, four of which were by cesarean sections (a fifth patient also had a previously planned cesarean with complications postoperatively). Based on the observations in this subgroup, with only two patients testing positive for pathogenic variants in our study, it is hypothesized that patients with a severe genetic predisposition do not necessarily need a severe trigger to develop aHUS, whereas patients with a more severe trigger might not need a known strong pathogenic variant in order to develop the disease.

The proportion of treatment-related AEs in the full 311 cohort was similar to that of the subgroup of patients in this analysis (34.5% and 37.5%, respectively) and no additional safety concerns were identified here. In the full 311 cohort the most common AEs were headache, diarrhea, and vomiting, while headache and pyrexia were the most common in this subgroup. No patients died or contracted a meningococcal infection.

E. Conclusions

In this first prospective interventional trial assessing the efficacy and safety of the long-acting C5 inhibitor ULTOMIRIS® (ravulizumab), TMA caused by aHUS was rapidly resolved in postpartum patients, with continued improvement over time and an acceptable safety profile. In addition, treatment with ULTOMIRIS® (ravulizumab) at 8-weekly dosing intervals resulted in rapidly improved hematological and renal endpoints in patients with postpartum-aHUS. The results from this study support the use of ULTOMIRIS® (ravulizumab) in women presenting with aHUS postpartum.

### Example 2: A Global aHUS Registry Analysis: Characteristics and Outcomes of Pregnancy-Triggered Atypical Hemolytic Uremic Syndrome (aHUS)

Pregnancy-triggered aHUS (p-aHUS) accounts for 10-20% of aHUS diagnoses. Complement-mediated thrombotic microangiopathy (CM-TMA) may be associated with high maternal and fetal morbidity and mortality, including end-stage renal disease (ESRD). The clinical characteristics of p-aHUS and survival probability in patients treated with the complement C5 inhibitor eculizumab were described, using the largest collection of p-aHUS data available in a single study.

A. Methods

Patients with a clinical diagnosis of aHUS were included in the global aHUS registry (NCT01522183). Patients with p-aHUS were identified as those with first TMA manifestations during pregnancy or within 60 days postpartum. Patients with other triggers of aHUS were excluded. Survival, based on time to ESRD, was calculated by the Kaplan-Meier method.

B. Results

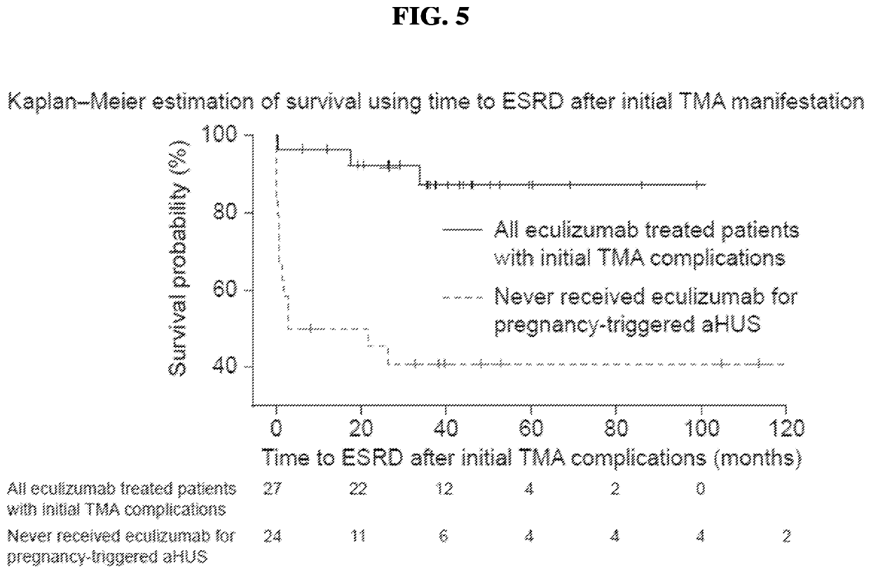

In the registry, 51/1029 female patients were diagnosed with p-aHUS and 27 received eculizumab. Mean±SD age at pregnancy onset was 30.7±5.9 years. p-aHUS occurred during pregnancy in 28 (54.9%) patients, with the remainder occurring postpartum. A diagnosis of pre-eclampsia or HELLP (hemolysis elevated liver enzymes low platelet count) syndrome was reported in 28 (54.9%) and 17 (33.3%) patients, respectively. A complement pathogenic variant was identified in 23 (45.1%) patients, of whom 3 (8.3%) also tested positive for anti-complement factor H antibodies. Mean±SD eculizumab treatment duration was 1.8±1.8 years. Survival probability was higher in eculizumab-treated patients compared with patients not receiving eculizumab (see FIG. 5).

C. Discussion

Survival probability was lower in patients diagnosed when eculizumab was unavailable, compared with patients who received eculizumab. Successful treatment with eculizumab, in addition to almost half of the patients having a complement pathogenic variant, confirms the appropriate classification of P-aHUS as a CM-TMA.

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYC
ARYFFGSSPNWYFDVWGQGTLVTVSS
SEQ ID NO: 8
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK
SEQ ID NO: 9
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
SEQ ID NO: 10
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNF
GTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
SEQ ID NO: 11
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 12
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHSHYTQKSLSLSLGK
SEQ ID NO: 15
ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
SEQ ID NO: 16
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGG
IGPFFGTANYAQKFQGRVTITADESTSTAYMELSSLRSEDTAVYYCARDT
PYFDYWGQGTLVTVSS
SEQ ID NO: 28
DIELTQPPSVSVAPGQTARISCSGDSIPNYYVYWYQQKPGQAPVLVIYDD
SNRPSGIPERFSGSNSGNTATLTISGTQAEDEADYYCQSFDSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGII
DPDDSYTEYSPSFQGQVTISADKSISTAYLQWSSLKASDTAMYYCARYEY
GGFDIWGQGTLVTVSS
SEQ ID NO: 36
SYELTQPPSVSVAPGQTARISCSGDNIGNSYVHWYQQKPGQAPVLVIYKD
NDRPSGIPERFSGSNSGNTATLTISGTQAEDEADYYCGTYDIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVG
AIYTGSGATYKASWAKGRFTISKDTSKNQVVLTMTNMDPVDTATYYCASD
GGYDYPTHAMHYWGQGTLVTVSS
SEQ ID NO: 44
DVVMTQSPSSLSASVGDRVTITCQASQNIGSSLAWYQQKPGQAPRLLIYG
ASKTHSGVPSRFSGSGSGTDFTLTISSLQPEDVATYYCQSTKVGSSYGNH
FGGGTKVEIK
SEQ ID NO: 45
QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P
SEQ ID NO: 46
DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
SEQ ID NO: 47
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSS
SEQ ID NO: 48
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIK
SEQ ID NO: 49
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

What is claimed is:

1. A method of treating a human patient with pregnancy-associated atypical haemolytic uraemic syndrome (p-aHUS), the method comprising administering to the patient an effective amount of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively.

2. The method of claim 1, wherein the antibody comprises a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fe region, each in EU numbering.

3. The method of claim 1 or 2, wherein the anti-C5 antibody, or antigen binding fragment thereof, comprises a heavy chain variable region as set forth in SEQ ID NO:12 and a light chain variable region as set forth in SEQ ID NO:8.

4. The method of any one of claims 1-3, wherein the anti-C5 antibody, or antigen-binding fragment thereof, further comprises a heavy chain constant region depicted in SEQ ID NO:13.

5. The method of any one of claims 1-4, wherein the anti-C5 antibody, or antigen binding fragment thereof, comprises a heavy chain as set forth in SEQ ID NO:14 and a light chain as set forth in SEQ ID NO:11.

6. The method of any one of claims 1-5, wherein the anti-C5 antibody, or antigen-binding fragment thereof, binds to human C5 at pH 7.4 and 25° C. with an affinity dissociation constant (K_{D}) that is in the range 0.1 nM≤K_{D}≤1 nM.

7. The method of any one of claims 1-5, wherein the anti-C5 antibody, or antigen-binding fragment thereof binds to human C5 at pH 6.0 and 25° C. with a K_{D}≥10 nM.

8. The method of any one of the preceding claims, wherein the anti-C5 antibody, or antigen binding fragment thereof, is formulated for intravenous administration.

9. The method of any one of the preceding claims, wherein the anti-C5 antibody, or antigen binding fragment thereof, is administered to the patient:
(a) once on Day 1 at a dose of: 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

10. A method of treating a human patient with p-aHUS, the method comprising administering to the patient an effective amount of an anti-C5 antibody, or antigen binding fragment thereof,
wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fc region, each in EU numbering, and
wherein the anti-C5 antibody, or antigen binding fragment thereof, is administered to the patient:
(a) once on Day 1 at a dose of: 2400 mg to a patient weighing ≥40 to <60 kg, 2700 mg to a patient weighing ≥60 to <100 kg, or 3000 mg to a patient weighing ≥100 kg; and
(b) on Day 15 and every eight weeks thereafter at a dose of 3000 mg to a patient weighing ≥40 to <60 kg, 3300 mg to a patient weighing ≥60 to <100 kg, or 3600 mg to a patient weighing ≥100 kg.

11. The method of any one of the preceding claims, wherein the anti-C5 antibody, or antigen binding fragment thereof, is administered to a patient weighing ≥40 to <60 kg:
(a) once on Day 1 at a dose of 2400 mg; and
(b) on Day 15 and every eight weeks thereafter at a dose of 3000 mg.

12. The method of any one of claims 1-11, wherein the anti-C5 antibody, or antigen binding fragment thereof, is administered to a patient weighing ≥60 to <100 kg:
(a) once on Day 1 at a dose of 2700 mg; and
(b) on Day 15 and every eight weeks thereafter at a dose of 3300 mg.

13. The method of any one of claims 1-11, wherein the anti-C5 antibody, or antigen binding fragment thereof, is administered to a patient weighing ≥100 kg:
(a) once on Day 1 at a dose of 3000 mg; and
(b) on Day 15 and every eight weeks thereafter at a dose of 3600 mg.

14. The method of any one of the preceding claims, wherein the treatment maintains a serum trough concentration of the anti-C5 antibody of 100 μg/ml or greater during the treatment.

15. The method of any one of the preceding claims, wherein the treatment maintains a serum trough concentration of the anti-C5 antibody of 200 μg/ml or greater during the treatment.

16. The method of any one of the preceding claims, wherein the treatment reduces free C5 concentration by greater than 99% throughout the treatment period.

17. The method of any one of the preceding claims, wherein the anti-C5 antibody, or antigen binding fragment thereof, is administered at a dose of 3000 mg, 3300 mg, or 3600 mg every eight weeks after the treatment for up to two years.

18. The method of any one of the preceding claims, wherein the treatment results in terminal complement inhibition.

19. The method of any one of the preceding claims, wherein the treatment results in a reduction of hemolysis compared to baseline as assessed by lactate dehydrogenase (LDH) levels.

20. The method of any one of the preceding claims, wherein the treatment results in a normalization of LDH levels.

21. The method of any one of the preceding claims, wherein the treatment produces a shift toward normal levels of a hemolysis-related hematologic biomarker selected from the group consisting free hemoglobin, haptoglobin, reticulocyte count, PNH red blood cell (RBC) clone and D-dimer.

22. The method of any one of the preceding claims, wherein the treatment produces at least one therapeutic effect selected from the group consisting of a reduction or cessation in severe hypertension, proteinuria, uremia, lethargy, fatigue, irritability, thrombocytopenia, microangiopathic hemolytic anemia, and renal function impairment compared to baseline.

23. The method of any one of the preceding claims, wherein the treatment produces a shift toward normal levels of Factor Ba, soluble tumor necrosis factor receptor 1 [sTNFR1]), soluble vascular adhesion molecule 1 [sVCAM1], thrombomodulin, D-dimer, and cystatin C.

24. The method of any one of the preceding claims, wherein the treatment produces an increase in hemoglobin stabilization compared to baseline.

25. The method of any one of the preceding claims, wherein the treatment produces a reduction in the need for blood transfusions compared to baseline.

26. The method of any one of the preceding claims, wherein the treatment produces a reduction in major adverse vascular events (MAVEs).

27. The method of any one of the preceding claims, wherein the treatment produces a change from baseline in quality of life, as assessed via the Functional Assessment of Chronic Illness Therapy (FACIT)-Fatigue Scale, version 4 and the European Organisation for Research and Treatment of Cancer, Quality of Life Questionnaire-Core 30 Scale.

28. The method of any one of the preceding claims, wherein the treatment results in platelet normalization.

29. The method of any one of the preceding claims, wherein the treatment results in a ≥25% improvement from baseline in serum creatinine.

30. The method of any one of the preceding claims, wherein the results in a complete TMA response.

31. The method of any one of the preceding claims, wherein the treatment results in a modified complete TMA response.

32. The method of any one of the preceding claims, wherein the treatment results in a shift towards normal levels of eGFR (e.g., 90).

33. The method of any one of the preceding claims, wherein the treatment results in a reduction or discontinuation of dialysis.

34. The method of any one of the preceding claims, wherein the treatment prevents or prolongs time to end-stage renal disease (ESRD).

35. The method of any one of the preceding claims, wherein the treatment prolongs survival of the patient.

36. The method of any one of the preceding claims, wherein the p-aHUS is postpartum aHUS.

37. A kit for treating pregnancy-associated atypical haemolytic uraemic syndrome (p-aHUS) in a human patient, the kit comprising:
(a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively; and
(b) instructions for using the anti-C5 antibody, or antigen binding fragment thereof, in the method of any one of claims 1-32.

38. A kit for treating pregnancy-associated atypical haemolytic uraemic syndrome (p-aHUS) in a human patient, the kit comprising:
(a) a dose of an anti-C5 antibody, or antigen binding fragment thereof, wherein the anti-C5 antibody, or antigen binding fragment thereof, comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fe region that binds to human neonatal Fc receptor (FcRn), wherein the variant human Fc CH3 region comprises Met-429-Leu and Asn-435-Ser substitutions at residues corresponding to methionine 428 and asparagine 434 of a native human IgG Fc region, each in EU numbering; and
(b) instructions for using the anti-C5 antibody, or antigen binding fragment thereof, in the method of any one of claims 1-32.

39. The kit of claim 35 or 36 wherein the p-aHUS is postpartum aHUS.


SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC